(19) United States
(12) Patent Application Publication
(10) Pub. No.: US 2007/0191320 A1
(43) Pub. Date: Aug. 16, 2007

(54) METHODS OF TREATMENT FOR FEMALE SEXUAL AROUSAL DISORDER

(75) Inventors: James L. Yeager, Lake Forest, IL (US); Mingql Lu, Plainsboro, NJ (US)

Correspondence Address: MIRICK, O'CONNELL, DEMALLIE & LOUGEE, LLP, 1700 WEST PARK DRIVE, WESTBOROUGH, MA, 01581, US

(73) Assignee: NexMed Holdings, Inc.

(21) Appl. No.: 11/618,722

(22) Filed: Dec. 29, 2006

Related U.S. Application Data
(63) Continuation-in-part of application No. 10/855,280, filed on May 27, 2004, now abandoned.
Application No. 10/855,280 is a continuation-in-part of application No. 10/188,554, filed on Jul. 2, 2002, now Pat. No. 6,825,234.
Application No. 10/188,554 is a continuation-in-part of application No. 09/208,965, filed on Dec. 10, 1998, now Pat. No. 6,486,207.

(51) Int. Cl. A61K 31/56; A61K 31/557

(52) U.S. Cl. 514/170; 514/573

(57) ABSTRACT

The invention provides methods of treatment for female sexual arousal disorder. In preferred embodiments, the invention provides methods of treatment with a semisolid composition suitable for topical application comprising: an effective amount of a vasoactive prostaglandin, a penetration enhancer, a polymer thickener, a lipophilic component, water and an acidic buffer system.

### CROSS REFERENCES TO RELATED APPLICATIONS

The present application is a continuation-in-part of U.S. patent application Ser. No. 10/855,280, filed May 27, 2004, which is a continuation-in-part of U.S. patent application Ser. No. 10/188,554, filed Jul. 2, 2002, now issued U.S. Pat. No. 6,825,234, which is a continuation-in-part of U.S. patent application Ser. No. 09/208,965 filed Dec. 10, 1998, now issued U.S. Pat. No. 6,486,207, which is related to International Application No. PCT/US99/29471, filed Dec. 10, 1999. The entire contents of the above applications are incorporated herein by reference.

### BACKGROUND OF THE INVENTION

Sexual dysfunction has been a persistent problem, more frequent in an aging population that has only recently been addressed with frank evaluation, scientific investigation and effective treatment. Male impotence, especially male erectile dysfunction, has received the most attention. Female sexual dysfunction has been considered in the context of male erectile dysfunction, in part because of the anatomical and physiological parallels between the male and female genitalia, and in part, with the hope that effective treatments for male erectile dysfunction could provide some relief for female sexual dysfunction.

Both male and female sexual behavior is viewed from the standpoint of a four-phase sexual response cycle consisting of the stages of desire, excitement, orgasm and resolution. Studies have shown that while there are many similarities between male and female sexual response, significant differences exist. Specific dysfunctions have been correlated with the phases of the model. The female sexual response and its dysfunctions remain poorly understood.

Female sexual arousal disorder (FSAD) is the persistent or recurrent inability to attain, or to maintain, sufficient sexual excitement, which causes personal distress. It may be expressed as lack of subjective excitement, lack of genital response, such as lubrication and swelling, or lack of other somatic responses. Female sexual arousal disorder is one form of female sexual dysfunction, and is associated with the excitement phase. See Basson, R., et al., Report of the international consensus development conference on female sexual dysfunction: definitions and classifications, J. Urol. 2000; 163(3):888-93.

While increased understanding of the pathophysiology of male erectile dysfunction has progressed rapidly in the past decade and led to new therapeutic modalities, little has been done to address similar issues in women. Cardiovascular risk factors have been shown to correlate with complaints of vaginal and clitoral dysfunction. Goldstein, M. K., et al., Gynecological factors in sexual dysfunction of the older woman. Clin Geriatr Med 7: 41-61, (1991); Sadeghi-Nejad, H., et al.: Impotence is a couple's disease: studies in female sexual dysfunction. J Urol 155: 677A, (1996); Slob, A. K., et al.: Sexuality and psychophysiological functioning in women with diabetes mellitus. J Sex Marital Ther: 59-69, (1990).

The correlation of cardiovascular risk factors and complaints of vaginal and clitoral dysfunction have led to suggestions that a significant degree of female sexual dysfunction is due to vascular insufficiency and therefore amenable to treatment with vasoactive agents. The underlying foundations of the normal and dysfunctional female sexual response must be considered in the context of the anatomy and physiology, summarized below. See, generally, Goldstein, I., and Berman, J. R., Vasculogenic female sexual dysfunction: vaginal engorgement and clitoral erectile insufficiency syndromes, Int. J. Impotence Research 10: Suppl. 2, S84-S90 (1998).

Anatomy of the Vagina

The vagina is the canal that connects the uterus with the external genital organs. Its design easily accommodates penetration of a rigid penile erection. At the posterior end the rounded neck of the uterus, the cervix, projects into the space known as the formix or vaginal vault. Anteriorly, two pleats of sensitive tissue, the labia minora, surround the opening of the vagina and are further protected by larger folds known as the labia majora.

The walls of the vagina consist of three layers—an inner mucosa, an aglandular mucous membrane epithelium, an intermediate, highly vascularized muscularis layer, and an outer supportive fibrous mesh. The vaginal mucosa is a mucous type stratified squamous cell epithelium that undergoes hormone-related cyclical changes, such as a slight keratinization of the superficial cells during the menstrual cycle. The muscularis portion comprises smooth muscle and an extensive arborization of blood vessels that may swell during intercourse. The surrounding fibrous layer provides structural support to the vagina; this layer consists of elastin and collagen fibers that allow for expansion of the vaginal vault during sexual arousal or childbirth. Large blood vessels run within the mucosa, and nerve plexuses are present within muscular and adventitial layers. The vagina has many rugae or folds that are necessary for the distensibility of the organ during intercourse and childbirth. Smaller ridges lend to the frictional tension that exists during intercourse.

The arterial supply to the vagina is derived from an extensive network of branching vessels surrounding it from all sides. The anterior branch of the internal iliac artery continually bifurcates as it descends through the pelvis with a series of the newly generated vessels, each supplying the vagina to some degree. After giving off an obturator artery branch, the umbilical, and the middle rectal arteries diverge off to supply a superior and inferior vesical artery, respectively. Between the umbilical and the mid-rectal branches there is a generation of a uterine artery, which further bifurcates to give the vaginal artery. The internal pudendal and accessory pudendal artery also send a branch to the vaginal artery. Finally, the common clitoral artery sends a branch to the vaginal muscularis.

The neurologic innervation of the vagina originates from two separate plexuses, the superior hypogastric plexus and the sacral plexus, The hypogastric nerve plexus descends on the great vessels spreading into an inferior hypogastric plexus, which systematically branches further into a uterovaginal nerve. The somatic pudendal nerve originates off the pelvic splanchnic branches from the secret plexus. Pudendal branching innervates the vagina towards the opening of the introitus as the perineal and posterior labial nerves.

Immunohistochemistry studies have been utilized to better understand the innervation of the human vaginal mucosa. In a study by Hilliges et al. using protein gene product 9.5, more distal areas of the vagina had significantly more nerve fibers compared to the more proximal parts, and the anterior wall showed a denser innervation than the posterior wall (Hilliges, M. et al., Innervation of the human vaginal mucosa as revealed by PGP 9.5 immunohistochemistry, Acta Anatomica 153: 119 (1995)). Graf et al studied the distribution patterns and the occurrence of helospectin and pituitary adenylate cyclase activating polypeptide (PACAP) immunoreactivity (Graf, A. H., et al. Helospectin and pituitary adenylate cyclase activating polypeptide in the human vagina, Regul. Pept. 55: 277 (1995)). They confirmed a dense network of vasoactive intestinal peptide (VIP) immunoreactive nerve fibers showing sub-populations of helospectin and LI-type PACAP. Nerve fibers of the vagina had previously been shown to be active in association with specific peptides that include VIP, peptide histidine methionine (PHM), calcitonin gene related peptide (CGPP), and galanin. Genital vasodilation and subsequent increase in vaginal blood flow and lubrication have been observed upon exposure of vessels to VIP. VIP has been implicated as the neurotransmitter for mediating vaginal vasodilation and the formation of lubricating fluid during sexual arousal. Helospectin and PACAP, a potent vasodilator, belong to the same peptide family as VIP and PHM, and recent observations have been made to the effect that distributions and co-localizations of helospectin and VEP as well as PACAP and VIP have been reported in the mammalian gastrointestinal tract.

The vaginal canal is lubricated primarily from a transudate originating from the subepithelial vascular bed passively transported through the interepithelial spaces, sometimes referred to as intercellular channels. Additional moistening during intercourse comes from secretion of the paired greater vestibular or Bartholin's glands.

Estrogen effects on the maintenance and function of female genitalia have been well documented in studies. Estrogen receptors have been shown to exist throughout the vaginal epithelium, in stromal cells, and in the smooth muscle fibers in the muscularis. Weaker conformations of estrogen such as estriol appear more effective in stimulating the vagina as opposed to the uterus. Thickness and rugae of the vaginal wall, as well as vaginal lubrication, have been shown to be estrogen dependent. Although this fluid production has been shown to be hormone-dependent both in the resting state and during sexual excitement, quantitative changes apparently do not occur during the menstrual cycle. An insufficient amount of estrogen will result in thin vaginal walls more easily susceptible to trauma with a decreased ability to heal, as well as a drier and less acidic vaginal environment more vulnerable to infection. Vaginal dryness is associated with ovarian failure and is effectively controlled by estrogen replacement therapy. Some women who are not sexually active may not notice the extent of vaginal atrophy but when coitus does resume, pain and discomfort from intercourse can be considerable.

Anatomy of the Clitoris

The clitoris is the homologue of the penis arising from the embryological genital tubercle. The clitoris consists of a cylindrical, erectile organ composed of three parts: the outermost glans or head, the middle corpus or body, and the innermost crura. The glans of the clitoris is visualized as it emerges from the labia minora, which bifurcate to form the upper prepuce anteriorly and the lower fronulum posteriorly. The body of the clitoris consists of two paired corpora cavernosa of about 2.5 cm in length and lacks a corpus spongiosum. The body extends under the skin at the corona to the crura. The two crura of the clitoris, formed from the separation of the most proximal portions of the corpora in the perineum, attach bilaterally to the undersurface of the symphysis pubis at the ischiopubic rami. A fibrous tunica albuginea ensheathes each corporal body made up of lacunar space sinusoids surrounded by trabecula of vascular smooth muscle and collagen connective tissue. No retractor clitoridis muscle exists in humans as it does in other animals such as cattle and sheep, however a supporting suspensory ligament does hold the clitoris in the introital region.

The main arterial supply to the clitoris is from the illo-hypogastric-pudendal arterial bed. The internal pudendal artery is the last anterior branch off the internal iliac artery. Distally, the internal pudendal artery traverses Alcock's canal, a position of the obturator fascia and lies on the inner side in apposition to the ischio-pubic ramus. In this latter location, the artery is susceptible to blunt perineal trauma. The internal pudendal artery terminates as it supplies the inferior rectal and perineal artery, which supplies the labia. The common clitoral artery continues to the clitoris. This artery bifurcates into a dorsal clitoral artery and a cavernosal clitoral artery.

Autonomic efferent innervation of the clitoris passes from the pelvic and hypogastric nerves to the clitoris through the urogenital diaphragm. Pelvic nerve stimulation results in clitoral smooth muscle relaxation and arterial smooth muscle dilation. There is a rise in clitoral cavernosal artery inflow, an increase in clitoral intracavernous pressure which lead to tumescence and extrusion of the glans clitoris.

Anatomical studies using female rats have indicated that the major neuronal input to the clitoris was seen in spinal segments from L5-S1, and to a lesser extent in T12-L4 as well as S2-S4. When a label that is taken up by nerve terminals and transported retrogradely to the nerve cell bodies (pseudorabies virus) was injected into the clitoris, labeled nerve cell bodies were found in the brain in multiple locations, including the nucleus paragigantocellularis, raphe pallidus, raphe magnus, Barrington's nucleus, ventrolateral central gray, hypothalamus, and the medial pre-optic region. This implies a multisynaptic circuit of neurons may be involved in clitoral neurological control rather than just a simple somatic reflex connection.

Morphological studies have been performed using wheat germ agglutinin conjugated with horseradish peroxidase (WGA/HRP) injected into the clitoris of the female cat to compare afferent pathways to the entire population of pudendal nerve afferents. Central projections of the clitoral afferents were identified in the L7-S3 segments with the most prominent labeling in S1-S2. In the same study, electrophysiological analysis of the clitoris performed under constant mechanical pressure stimulation indicated both phasic and tonic discharges in L7-S2, but most prominently in S1. In contrast electrical stimulation of the clitoris evoked discharges at S1 only. The neurotransmitters mediating clitoral and arterial smooth muscle dilation remain undetermined, however preliminary studies suggest that nitric oxide is involved. Histochemical studies have revealed VIP and neuropeptide Y (NPY) immunoreactive nerves in the clitoral erectile tissues. Somatic sensory pathways originate from the clitoral skin. There exists a dense collection of Pacinian corpuscles innervated by rapidly adapting myelinated afferents, as well as Meissner's corpuscles, Merckel tactile disks, and free nerve endings. These sensory afferents pass from the dorsal clitoral nerve to the pudendal nerve.

The Grafenberg Spot

The Grafenberg spot (or G-spot) can also play a role in female sexual arousal. The current information regarding the Grafenberg zone (also known as Grafenberg spot, or G-spot) was recently summarized (Goldstein, I., et al., “Female Sexual Dysfunction” pp. 507-557, at 523 in Jardin, A, et al., editors, Erectile Dysfunction, (First International Consultation on Erectile Dysfunction, co-sponsored by the World Health Organization (WHO), International Consultation on Urological Diseases (ICUD) and Societe Internationale d'Urologie (SIU), held Jul. 1-3, 1999, Paris. 2000). Grafenberg reported that the digital stroking of the anterior vagina along the urethra, especially in the region of the base of the bladder, sexually aroused female subjects greatly (Grafenberg E. (1950): The role of the urethra in the female orgasm. Int. J. Sexology. 3: 145-148). In a number of women this region swelled up to the size of a kidney bean and projected into the vaginal lumen. Few took any notice of this finding. The area was rediscovered and renamed the G-spot in honor of Grafenberg (Ladad, A. K., et al., (1982): The G spot and other recent discoveries about Human Sexuality. Holt, Rinehart & Winston, New York). Other investigators could not locate a “spot” but found, rather than a punctate locus, a general excitable area along the whole length of the urethra running along the anterior vaginal wall (Hoch Z. (1986): Vaginal erotic sensitivity by sexological examination. Acta Obstet. et Gynecol. Scand. 65: 768-773). When this was stimulated manually, the sexual arousal induced was almost immediate. Alzate & Londono located the erotic sensitive area in closer relation to the bladder base than the urethra (Alzate H. & Londono M. L. (1984): Vaginal erotic sensitivity. J. Sex & Marital Therapy. 10: 49-56). Lenck, et al. localized by ultrasound in the living subjects the underlying structure in the anterior vaginal wall that gave the erotic sensations on stimulation as the urethral sphincter confirming it by dissection in the cadaver (Lenck L. Ch., et al., (1992): Sphincter uretral (point G) correlations anatomo-cliniques. Revue Francais de Gyncologie et Obstrique. 87: 65-69.). Other investigators have implied that the G spot/area represents that part of the urethra that contains the periglandular or paraurethral tissue, corresponding to the female equivalent of the prostate (See Zaviacic M. & Whipple B. (1993): Update on the female prostate and the phenomenon of female ejaculation. J. Sex Research. 30: 148-151, for references). These glands are present to a greater or lesser degree in about 90% of women.

Physiology of Female Sexual Arousal

The female sexual response phase of arousal is not easily distinguished from the phase of desire until physiological changes begin to take place in the vagina and clitoris as well as other sexual organs. Sexual excitement and pleasure are accompanied by pelvic vasocongestion and swelling of the external genitalia including vaginal engorgement and clitoral erection.

Vaginal engorgement enables a process of plasma transudation to occur, allowing a flow through the epithelium and onto the vaginal surface. Plasma transudation results from the rising pressure in the vaginal capillary bed during the arousal state. In addition there is an increase in vaginal length and luminal diameter, especially in the distal 2/3 of the vaginal canal.

Dissociation of Genital Reflexes from Subjective Arousal

Central nervous system areas primarily implicated in sexual arousal, based on animal research, include the medial preoptic, anterior hypothalamic region and related limbic-hippocampal structures. Cognitive effects have been investigated, and in one study the results suggest that the greatest contribution to sexual arousal in the female results from cognitive processing of stimulus content and meaning, and not from peripheral vasocongestive feedback (Laan, E., et al., Determinants of subjective experience of sexual arousal in women. Feedback from genital arousal and erotic stimulus content, Psychophysiol. 32: 44-(1995)).

The distinction between local physiological aspects of sexual response, such as genital vasocongestion measured by vaginal photoplesmography, and subjective sexual arousal, measured by self-reporting rating scales and inventories has been clearly demonstrated in both normal and sexually dysfunctional women (Palace, E. M. and Goralka, B. B., Differential patterns of arousal in sexually functional and dysfunctional women: Physiological and subjective components of sexual response, Arch. Sexual Behav. 21: 135-159 (1992)). Several reliable and validated self-report inventories are recognized for measurement of female sexual function (Derogatis, L. R. and Conklin-Powers, B., Psychological assessment measures of female sexual functioning in clinical trials, Int. J. Impot. Res. 10 Suppl. 2: S111-S116 (1998)).

There does not appear to be a relation between menstrual phases and physiologic arousability. Meuwissen and Over (Habituation and Dishabituation of Female Sexual Arousal, Behav. Res. Ther. 28: 217-(1990)) have found that neither film-induced nor fantasy-induced levels of sexual arousal varied significantly throughout the menstrual cycle. There are conflicting reports as well as to the habituation of the female sexual response. Some claim that levels of subjective and physiologic sexual arousal decrease over repeated exposure to sexual stimuli. Others could not elucidate similar results even after 21 trials, yet both concur that the subsequent presentation of a novel stimulus will increase the female sexual response. The desire for increased sexual performance on sexual arousal in functional women have been found to facilitate genital responses, most prominently with the stimulus of erotic fantasy as opposed to erotic film. Interestingly, masturbation frequency had no affect on genital responses despite its significance on subjective reports of arousal. (Laan et al, 1995; Meuwissen and Over, 1990).

Clinicians and researchers have assumed that sexual arousal, is inhibited by the sympathetic nervous system, while facilitation and maintenance are through the parasympathetic nervous system. However, studies have challenged these notions in the woman. Intense exercise, consisting of twenty-minute bike riding sessions, increased physiological sexual arousal measured by vaginal photoplethysmography. This challenged the notion that sympathetic nervous system stimulation inhibited sexual arousal in women and further provided evidence that sexual arousal was actually facilitated by the sympathetic nervous system. Another study examined the temporal effect of sympathetic activation through acute exercise on immediate delayed, and residual sexual arousal. Sexual arousal was objectively assessed by vaginal plethysmography. A relationship between sympathetic nervous system activation and sexual arousal was found, such that sexual arousability was inhibited five minutes post-exercise and was facilitated fifteen minutes post-exercise and only marginally increased thirty minutes post-exercise. The two studies suggest that sympathetic nerve stimulation activation plays an important facilitatory role in the early stages of sexual arousal.

The clitoris may play a major role during sexual activity in that it is not only part of what makes the sexual act enjoyable for the woman but also enhances her response to coitus upon clitoral stimulation. Clitoral stimulation may induce local autonomic and somatic reflexes causing vaginal vasocongestion, engorgement, and subsequent transudation, lubricating the introital canal making the sexual act easier, more comfortable, and more pleasurable. The more stimulation, the higher the level of arousal and the easier it is to further increase stimulations.

Vasculogenic Female Sexual Dysfunction

Female sexual dysfunction has traditionally included disorders of desire/libido, disorders of arousal, pelvic pain disorders, and inhibited orgasm. Patient surveys estimate that 18-76% of adult women have such complaints during sexual activity. Female sexual dysfunction which may have its origin in abnormal arterial circulation into the vagina or clitoris during sexual stimulation, usually from atherosclerotic vascular disease may be considered a disorder of arousal. This vasculogenic female sexual dysfunction may include such clinical symptoms as delayed vaginal engorgement, diminished vaginal lubrication, pain or discomfort with intercourse, diminished vaginal sensation, diminished vaginal orgasm, diminished clitoral sensation or diminished clitoral orgasm. Traumatic injury to the ilio-hypogastric-pudendal arterial bed from pelvic fractures or blunt perineal trauma may also result in diminished vaginal/clitoral blood flow following sexual stimulation and fall into this vasculogenic category.

Prostaglandins

The prostaglandins are a series of cyclic derivatives of certain unsaturated fatty acids. They are found in a variety of tissues, including the prostate gland, the seminal vesicles, the lungs and the brain. These naturally occurring prostaglandins are derived by cyclization of 20-carbon unsaturated fatty acids such as arachidonic acid. See Lehninger, Albert L., Biochemistry, 2d ed. (1975), p. 300 (hereinafter “Lehninger”).

Prostaglandins as a class of compounds have diverse pharmacologic activity, including stimulation of gastrointestinal and reproductive smooth muscle, relaxation and contraction of respiratory smooth muscle, hypotensive activity, inhibition of fatty acid lipolysis, inhibition of blood platelet aggregation, and inhibition of gastric acid secretion. Therapeutic utility of prostaglandins in general is correspondingly broad. As for prostaglandin E₁ (“PGE₁”) in particular, this compound, salts thereof, and lower alkyl esters thereof are well known and disclosed, e.g., in U.S. Pat. Nos. 3,069,322 (Bergstrom et al.), 5,219,885 (Froelich et al.) and in J. Org. Chem. 1974, 37, 2921. PGE₁ has found utility in the treatment of peripheral occlusive diseases, acute myocardial infarction, angina pectoris, acute ischemic stroke, asthma, gastrointestinal ulcers, ulcers of the skin, and organ rejection. Various routes of administration have been described, including oral, intravenous, buccal, rectal, intra-arterial, subcutaneous, and sublingual. The preferred route of administration of PGE₁ will of course be dependent on the particular intended therapeutic use.

Prostaglandins are well known to those skilled in the art. This class of drugs includes those derivatives of prostanoic acid (5-octylcyclopentaneheptanoic acid) referred to as A-I series prostaglandins. Prostaglandin nomenclature is well known and disclosed, e.g., in page 409, Remington's Pharmaceutical Sciences, 18th Edition, 1990, A. R. Gennaro, Ed., Mack Publishing Company, Easton, Pa. The term “prostaglandin” as used generically herein refers to the prostaglandin free acid and pharmaceutically acceptable derivatives thereof, including PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{3α}, carboprost tromethamine, dinoprost tromethamine, dinoprostone, lipoprost, gemeprost, metenoprost, sulprostone and tiaprost as well as salts and esters thereof. Preferred prostaglandins for use in the formulations of this invention include those prostaglandins comprising a β-hydroxyketone moiety, including D-series and E-series prostaglandins, preferably E-series prostaglandins such as prostaglandin E₁, including pharmaceutically acceptable salts and lower alkyl esters thereof (the term “lower alkyl” as used herein means straight chain or branched chain alkyl containing one to four carbon atoms). Of the lower alkyl esters, the ethyl ester of prostaglandin E₁ (commercially available from Sigma Chemical Company, St. Louis, Mo., and preparable as disclosed, e.g., in U.S. Pat. No. 5,219,885, incorporated herein by reference) is preferred.

The biosynthesis of prostaglandins has been well characterized. See, e.g., Lehninger at p. 687. In a typical biosynthetic pathway, exemplified by production of PGE₂, the essential fatty acid linoleic acid is converted into the 20-carbon arachidonic acid, which is then acted upon by prostaglandin synthase, a dioxygenase enzyme. Oxygen atoms are added at carbon atoms 9 and 15, and the product is cyclized by formation of a bond between carbon atoms 8 and 12. In the presence of reduced glutathione, this cyclized product undergoes conversion into prostaglandin PGE₂. Other types of naturally occurring prostaglandins are derived from different polyunsaturated fatty acids.

In about the 1960s, prostaglandins were isolated from a particular species of Caribbean coral, which made them more widely available for research. Catanzarite, Valerian A. and Gary Aisenbrey, Contemporary OB/GYN (October 1987), p. 22. A large number of natural and synthetic analogues of the prostaglandins are now known. Lehninger at 687.

The prostaglandins are known to produce often unpredictable effects over a very wide range of biological activities of a hormonal or regulatory nature. Prostaglandins have been reported to both lower and raise blood pressure, to inhibit gastric secretion, dilate bronchi, inhibit lipolysis, antagonize vasopressin-induced anti-diarrhesis, constrict the pupil, increase and decrease the intraocular pressure and produce contraction of the uterus. See, e.g., Ganong, William F., Review of Medical Physiology, 7th ed. (1975), p. 226 (hereinafter “Ganong”). The naturally occurring prostaglandins all appear to be capable of affecting the control of vascular and other smooth muscle contractions. In the central nervous system, prostaglandins are known to modify responses to certain synaptic transmitters. They have been reported to mimic the actions of some hormones and to inhibit the actions of certain others. See Ganong at 226.

Two of the most extensively studied of the prostaglandins are PGE₂ and PGF_{2α}. Both of these molecules are synthesized within the pregnant and non-pregnant uterus. While PGE₂ and PGF_{2α} are similar in mediating some effects, they are different with respect to certain others. Both cause uterine contractions, but they predominate at different sites within the uterus—PGE₂ in the lower uterine segment, PGF_{2α} in the fundal region. Both play important roles during labor, but PGE₂ has its major effect in cervical ripening, whereas PGF_{2α} is more important in generating uterine contractions. PGE₂ elevates body temperature, whereas PGF_{2α} has no apparent effect on body temperature. PGE₂ is vasodilator and bronchodilator, while PGF_{2α} is a bronchoconstrictor and vasoconstrictor. See Catanzarite at 21-22.

Prostaglandins have been used in gynecology for pregnancy termination. Preparing the cervix with a prostaglandin suppository has been found to reduce the incidence of cervical laceration and significant bleeding. See Catanzarite at page 22. Synthetic analogues of prostaglandin PGE₂, such as 16-16-dimethyl PGE₂ and 9-methylene PGE₂, have proven useful for the induction of first trimester abortions. Such procedures typically use vaginal suppositories containing 20 milligrams PGE₂ or 3 milligrams of 15-methyl PGF_{2α}, or by repeated intramyometrial injections of 15-methyl PGF_{2α}, or by infusing a PGF_{2α}-urea mixture (20 milligrams of PGF_{2α} and 40 milligrams of urea in 100 mL of 5% dextrose in water) into the amniotic sac.

In obstetrics, prostaglandins have been used for cervical ripening, labor induction and control of post-partum hemorrhage. Catanzarite at 29. For cervical ripening, PGE₂ has been given intravenously, orally and vaginally, but the preferred route is intracervically. A PGE₂ gel is now commercially available in Scandinavia, and another PGE₂ gel is being investigated in the United States. The PGE₂ gel can also be used for labor induction (3-5 mg of PGE₂, prepared by blending a 20 mg suppository with 60 mL of lubricating jelly and using 9-15 mL of the mixture, is placed in the vagina). Catanzarite at 32. Prostaglandins have also been utilized to control post-partum hemorrhage.

Topical and transdermal drug formulations are designed to deliver a therapeutically effective amount of drug to or across the skin of a patient. Devices known to the art include reservoir type devices involving membranes that control the rate of drug release to the skin, gels and creams, and devices involving a dispersion of the drug in a matrix such as a pressure sensitive adhesive. As the skin presents a barrier to the drug it is often desirable or necessary to incorporate certain materials that enhance the rate at which the drug passes through the skin. For any particular drug, however, the type of device, the transdermal flux rate that is suitable, and suitable formulation components, are dependent upon the particular drug to be delivered.

Topical and transdermal administration of PGE₁ and PGE₁ derivatives have also been described, e.g., in U.S. Pat. Nos. 4,889,845 (Ritter et al.), 4,515,810 (Chow et al.), and 5,219,885 (Froelich et al.) and in Japanese Kokai 2-264725 (Morimoto et al.) and 63-135333 (Nakano et al.). In order for a transdermal formulation of PGE₁ or a derivative thereof to be effective and suitable it is desirable that the formulation have a high transdermal flux rate, allowing a therapeutically effective blood level of the drug to be achieved or maintained when the formulation is applied to a relatively small area of the skin. Furthermore PGE₁ readily undergoes certain reactions and rearrangements (see. e.g., J. Chromatography, 1991, 555, 73 (Lee et al.). This instability of the prostaglandin can be problematic in providing a suitable transdermal formulation.

### SUMMARY OF THE INVENTION

The present invention provides methods of treatment for female sexual arousal disorder. In preferred embodiments the present invention provides topical compositions and methods of treatment for female sexual arousal disorder. In preferred embodiments, the invention provides a method of treatment for female sexual arousal disorder comprising the steps of: providing a single dose of a topical prostaglandin composition comprising about 0.5 mg to about 1.5 mg prostaglandin E₁; administering the single dose of the topical prostaglandin composition to the clitoris and the inner anterior wall of the vagina about 5 to about 20 minutes before intercourse; and repeating the step of administering at least twice in six weeks. In other embodiments, the single dose of the topical prostaglandin composition comprises about 0.5 mg to about 0.9 mg prostaglandin E₁. In preferred embodiments, the administration is repeated at least three to five times in a month. In preferred embodiments, the topical composition of the present invention is applied to at least one, preferably both of two structures involved in female sexual arousal, the Grafenberg spot and the clitoris.

The composition of the invention is suitable for topical application, and comprises a vasoactive prostaglandin, more preferably prostaglandin E₁, a penetration enhancer, a polymer thickener, a lipophilic component, and an acidic buffer system. In some embodiments, the polymer thickener is a polyacrylic acid polymer. In other preferred embodiments, the polymer thickener is a polysaccharide gum or a modified polysaccharide gum. The lipophilic component is selected from the group consisting of the C₁ to C₈, aliphatic alcohols, the C₂ to C₃₀ aliphatic esters and mixtures thereof. The acidic buffer system is chosen to provide a suitable pH to minimize irritation of skin and mucous membranes. The composition is typically in the form of a cream, lotion, gel or other form suitable for topical application to skin and mucous membranes.

In a preferred embodiment, the present invention provides a composition suitable for topical application comprising an effective amount of a vasoactive prostaglandin; a penetration enhancer selected from the group consisting of an alkyl-(N-substituted amino) alkanoate, an alkyl-2-(N,N-disubstituted amino) alkanoate, an (N-substituted amino) alkanol alkanoate, an (N,N-disubstituted amino) alkanol alkanoate, pharmaceutically acceptable salts thereof and mixtures thereof; a polymer thickener selected from the group consisting of a polyacrylic acid polymer, a polysaccharide gum, a modified polysaccharide gum and mixtures thereof; a lipophilic component; and a buffer system wherein the pH of the composition is 3.0 to 7.4. Preferably the vasoactive prostaglandin is selected from the group consisting of prostaglandin E₁, prostaglandin E₁ alkyl esters, pharmaceutically acceptable salts thereof and mixtures thereof. In preferred embodiments, the composition further comprises polyethylene glycol.

### BRIEF DESCRIPTION OF THE DRAWINGS

In the drawings,

FIG. 1 is a graphical representation of the results of studies of Example 6 showing the primary efficacy measure, mean arousal success rate for the patients of the ITT population who made at least 3 sexual encounter attempts

FIG. 2 is a graphical representation of the results of studies of Example 6 showing the percent of women in each group who attempted intercourse at least 3 times out of the possible 10 times during the 6 week study and achieved satisfactory arousal during at least 50% of their attempts.

FIG. 3 is a graphical representation of the results of studies of Example 6 showing the mean change from baseline of the FSFI Arousal Domain.

FIG. 4 is a graphical representation of the results of studies of Example 6 showing efficacy results as measured by the Global Assessment Question (GAQ) “While on the study medication did you feel your sexual arousal improve?”

FIG. 5 is a graphical representation of the results of studies of Example 8 showing results from Question 3 of the FSEP, arousal success rate (Mean Percent of Attempts Resulting in Successful Arousal) for the four treatment groups.

FIG. 6 is a graphical representation of the results of studies showing of Example 8 showing the mean change from baseline of the FSFI total score for the four treatment groups.

FIG. 7 is a graphical representation of the results of studies of Example 8 showing the mean change from baseline of the FSDS score for the four treatment groups.

FIG. 8A is a graphical representation of the mean arousal success data of Table 22, where the error bars are ±1 standard error of the mean for the four treatment groups at the screening period, for the first treatment period, for the second treatment period and for the entire treatment period for the total ITT population.

FIG. 8B is a graphical representation of the mean arousal success data of Table 23, where the error bars are ±1 standard error of the mean for the four treatment groups at the screening period, for the first treatment period, for the second treatment period and for the entire treatment period for the postmenopausal patients.

FIG. 8C is a graphical representation of the mean arousal success data of Table 24, where the error bars are ±1 standard error of the mean for the four treatment groups at the screening period, for the first treatment period, for the second treatment period and for the entire treatment period for the premenopausal patients.

### DETAILED DESCRIPTION OF THE INVENTION

It must be noted that, as used in this specification and the appended claims, the singular forms “a”, “an” and “the” include plural referents unless the context clearly dictates otherwise. Thus, for example, reference to “a vasoactive agent” includes a mixture of two or more such drugs, reference to “a penetration enhancer” includes mixtures of two or more enhancers, and the like.

In describing and claiming the present invention, the following terminology will be used in accordance with the definitions set out below.

The term “drug” or “pharmacologically active agent” as used herein is intended to mean a compound or composition of matter which, when administered to an organism (human or animal) induces a desired pharmacologic and/or physiologic effect by local and/or systemic action. As noted above, the pharmacologically active agents used in conjunction with the present invention are vasoactive agents.

By “transdermal” drug delivery, applicant is using the term in its conventional sense, i.e., to indicate delivery of a drug by passage into and through the skin and the underlying tissues and into the blood stream. By “transmucosal” drug delivery, applicant intends delivery of a drug by passage of a drug through the mucosal and underlying tissue into the blood stream. The compositions, systems, and methods of the invention, unless explicitly stated otherwise, should be presumed to be equally applicable to either transdermal or transmucosal modes of drug delivery.

“Penetration enhancement” or “permeation enhancement” as used herein relates to an increase in the permeability of the skin or mucosal tissue to a selected pharmacologically active agent, i.e., so that the rate at which the drug permeates through the skin or mucosal tissue is increased. “Carriers” or “vehicles” as used herein refer to carrier materials suitable for transdermal or transmucosal drug administration, and include any such materials known in the art, e.g., any liquid, gel, solvent, liquid diluent, solubilizer, or the like, which is nontoxic and which does not interact with other components of the composition in a deleterious manner.

By an “effective” amount of a drug or pharmacologically active agent is meant a nontoxic but sufficient amount of the drug or agent to provide the desired effect.

In order to carry out the method of the invention, a composition suitable for topical application comprising a selected vasoactive agent is administered about fifteen minutes to about one hour prior to the time of desired effect. Preferably, the topical composition is applied once, twice or three times within a twenty-four hour period. In additional to the prostaglandin compositions described below, suitable topical prostaglandin compositions are also described in U.S. Pat. Nos. 6,046,244, 6,414,028, 6,693,135, 6,841,574 and U.S. Published Patent Application No. 20050181030, which are all incorporated by reference herein.

Suitable vasoactive agents include, but are not limited to: nitrates such as nitroglycerin, isosorbide dinitrate, erythrityl tetranitrate, amyl nitrate, sodium nitroprusside, molsidomine, linsidomine chlorhydrate and S-nitroso-N-acetyl-d,1-penicillamine (“SNAP”); long and short acting α-blockers such as phenoxybenzamine, dibenamine, doxazosin, terazosin, phentolamine, tolazoline, prazosin, trimazosin, alfuzosin, tamsulosin and indoramin; ergot alkaloids such as ergotamine and ergotamine analogs, e.g., acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride; antihypertensive agents such as diazoxide, hydralazine and minoxidil; vasodilators such as nimodepine, pinacidil, cyclandelate, dipyridamole and isoxsuprine; chlorpromazine; haloperidol; yohimbine; trazodone and vasoactive intestinal peptides. Prostaglandin E₁ and phentolamine are particularly preferred vasoactive agents for use in conjunction with the present method.

A dose of a prostaglandin E₁ in an amount sufficient to enhance engorgement or vaginal secretion is topically administered to a woman. The appropriate doses of the particular vasodilating agent may be readily determined using methods described in Examples 3 and 4, below. The female response may be measured using methods described in Masters, W. H. and Johnson, V. E., Human Sexual Response, Little, Brown, and Co., Boston (1966) which is incorporated herein by reference. Engorgement and redness of the external genitalia can be assessed by visual inspection. Methods for measuring blood flow, including Doppler ultrasonic velocimetry, thermography using for example an isothermal blood flow transducer, radioscintigraphic methods, vaginal photoplethysmography may be used as well as other methods well known in the art. In addition, measuring the contraction of the distal 1/3 as is characteristic of the plateau phase of female sexual response of the vagina may be measured using methods and equipment well known in the art including but not limited to strain gauges or other devices for measuring muscular contraction or muscle tension.

In addition, enhanced sexual response and heightened arousal can be measured using a questionnaire that requests that the female subject to describe any change in sensation brought about by administration of the prostaglandin composition by the methods of the present invention. In determining as suitable effective dose, appropriate placebo controls can be used to determine whether or not the observed effect is directly attributable to the administration of the prostaglandin composition. A suitable questionnaire for the measurement of enhanced sexual response and heightened arousal is provided below in Example 4.

A preferred embodiment of the present invention involves the topical administration of from at least 0.1 mg to about 6 mg of prostaglandin E₁ to a female from about one minute to about one hour before sexual intercourse, preferably about five minutes to about twenty minutes before sexual intercourse. In a preferred embodiment of the present invention, about 0.1 mg to about 2 mg of prostaglandin E₁ is administered topically. In another preferred embodiment of the present invention, about 0.5 mg to about 1.5 mg of prostaglandin E₁ is administered topically to a female. In another preferred embodiment of the present invention, about 0.5 mg to about 0.9 mg of prostaglandin E₁ is administered topically to a female.

A preferred composition comprises about 0.07 weight percent to about 0.4 weight percent of prostaglandin E₁ and a pharmaceutically acceptable excipient to form a composition suitable for topical application.

More particularly, in a preferred embodiment, the composition suitable for topical application of the present invention comprises:
a) about 0.07 percent by weight of the total composition to about 0.4 percent by weight of the total composition of prostaglandin E₁;
b) about 0.5 to about 5 percent by weight of the total composition of a suitable polymer;
c) about 70 to about 90 percent by weight of the total composition of a buffer;
d) about 0.5 to about 15 percent by weight of the total composition of a lipophilic component;
e) about 0.4 to about 5 percent by weight of the total composition of an emulsifier; and
f) about 50 to about 90 percent by weight of the total composition of water.

In one embodiment, the topical composition comprises 0.01 weight percent to 5 weight percent modified polysaccharide gum; 0.001 weight percent to 1 weight percent of a vasoactive prostaglandin selected from the group consisting of PGE₁, pharmaceutically acceptable salts thereof, lower alkyl esters thereof and mixtures thereof; 0.5 weight percent to 10 percent weight dodecyl N,N-dimethylamino isoproprionate or pharmaceutically acceptable salts thereof, 0.5 weight percent to 10 weight percent of a lower alcohol selected from the group consisting of ethanol, propanol, isopropanol and mixtures thereof, 0.5 weight percent to 10 weight percent of an ester selected from the group consisting of ethyl laurate, isopropyl myristate, isopropyl laurate and mixtures thereof, based on the total weight of the composition, and an acid buffer. In a preferred embodiment, the topical composition further comprises 1 weight percent to 25 weight percent, more preferably 3 weight percent to 20 weight percent polyethylene glycol 400, based on the total weight of the composition.

In addition, the present invention is concerned with a method for treating an human female suffering from sexual dysfunction. The method comprises administering an effective amount of the above disclosed topical composition by applying the topical composition to the genital area of a human female. In one embodiment of the present invention, the topical composition is applied to the labia, clitoris and the vulvar region of the vagina. In one embodiment of the present invention, the topical composition is applied to the vaginal Grafenberg spot (G-spot) and to the clitoris. In other embodiments, the composition of the present invention is applied to the anterior wall of the vagina, including the location of the G-spot and only optionally to the clitoris.

The stable, uniform, composition suitable for topical application of the present invention preferably contains prostaglandin as a vasoactive agent. Vasoactive prostaglandins are those that act as peripheral vasodilators, including naturally occurring prostaglandins such as PGE₁, PGA₁, PGB₁, PGF_{1α}, 19-hydroxy-PGA₁, 19-hydroxy-PGB₁, PGE₂, PGA₂, PGB₂, 19-hydroxy-PGA₂, 19-hydroxy-PGB₂, PGE₃, PGF_{2α} PGF_{3α}; semisynthetic or synthetic derivatives of natural prostaglandins, including carboprost tromethamine, dinoprost tromethamine, dinoprostone, lipoprost, gemeprost, metenoprost, sulprostone and tiaprost. Prostaglandin E₁ and prostaglandin E₂ are particularly preferred vasoactive prostaglandins for use in conjunction with the present method.

The quantity of vasoactive prostaglandin, such as prostaglandin E₁, in the pharmaceutical composition is a therapeutically effective amount and necessarily varies according to the desired dose, the dosage form (e.g., suppository or topical), and the particular form of vasoactive prostaglandin used. The term “prostaglandin” as used generically herein refers to the prostaglandin free acid and pharmaceutically acceptable derivatives thereof, including, for example PGE₁, pharmaceutically acceptable salts and lower alkyl esters thereof (the term “lower alkyl” as used herein means straight chain or branched chain alkyl containing one to four carbon atoms). The composition generally contains between 0.001 weight percent to 1 weight percent of vasoactive prostaglandin, e.g., prostaglandin E₁, typically contains between 0.05 weight percent to 1 weight percent, preferably from 0.1 weight percent to 0.5 weight percent, based on the total weight of the composition.

The amount that constitutes a therapeutically effective amount varies according to the particular prostaglandin to be delivered, the indication to be treated, the surface area of the skin and mucous membrane over which the formulation is to be placed, and on the other components of the composition. Accordingly it is not practical to enumerate particular preferred amounts but such can be readily determined by those skilled in the art with due consideration of these factors. Generally, however, the prostaglandin is present in an amount of about 0.07 to about 1 percent, preferably about 0.1 to about 1 percent by weight based on the total weight of the composition. In one preferred embodiment, prostaglandin E₁ is present in an amount of about 0.07 to about 0.4 percent by weight based on the total weight of the composition. The prostaglandin can be dissolved or substantially uniformly dispersed in the topical composition. It is preferably soluble (and dissolved) in the topical composition.

The topical composition can contain one or more penetration enhancers. Among the preferred penetration enhancers for the present invention are ethanol, propylene glycol, glycerol, ethyl laurate, isopropyl palmitate, isopropyl myristate, laurocapram (Azone™), dioxolanes (described in U.S. Pat. No. 4,861,764), macrocyclic ketones, HP-101, oxazolidones and biodegradable penetration enhancers (described in U.S. Pat. Nos. 4,980,378 and 5,082,866 to Wong et al. and U.S. Pat. No. 6,118,020 to Büyüktimkin et al. such as alkyl-2-(N-substituted amino) alkanoates (e.g., dodecyl N,N-dimethylamino isoproprionate (DDAIP)), N-substituted amino alkanol alkanoates), acid addition salts and mixtures thereof.

The penetration enhancer is present in an amount sufficient to enhance the penetration of the prostaglandin E₁. The specific amount varies necessarily according to the desired release rate and the specific form of prostaglandin E₁ used. Generally, the penetration enhancer is present in an amount ranging from about 0.5 weight percent to about 20 weight percent, based on the total weight of the composition. Preferably, the penetration enhancer is present in an amount ranging from about 1 weight percent to about 10 weight percent of the composition. More preferably, the penetration enhancer is present in an amount ranging from about 1 weight percent to about 5 weight percent of the composition.

Preferred penetration enhancers include an alkyl-2-(N-substituted amino)-alkanoate, a (N-substituted amino)-alkanol alkanoate, or a mixture of these. For convenient reference, alkyl-2-(N-substituted amino)-alkanoates and (N-substituted amino)-alkanol alkanoates can be grouped together under the term alkyl(N-substituted amino) esters.

Alkyl-2-(N-substituted amino)-alkanoates suitable for the present invention can be represented as follows:

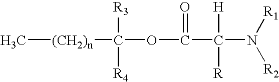

wherein n is an integer having a value in the range of about 4 to about 18; R is a member of the group consisting of hydrogen, C₁ to C₇ alkyl, benzyl and phenyl; R₁ and

R₂ are members of the group consisting of hydrogen and C₁ to C₇ alkyl; and R₃ and R₄ are members of the group consisting of hydrogen, methyl and ethyl.

Preferred are alkyl(N,N-disubstituted amino)-alkanoates such as C₄ to C₁₈ alkyl (N,N-disubstituted amino)-acetates and C₄ to C₁₈ alkyl(N,N-disubstituted amino)-propionates and pharmaceutically acceptable salts and derivatives thereof. Exemplary specific alkyl-2-(N,N-disubstituted amino)-alkanoates include dodecyl 2-(N,N dimethylamino)-propionate (DDAIP). For example, the preparation of crystalline acid addition salts of DDAIP by cooled mixing of DDAIP with one of a select group of acids in the presence of a water-immiscible solvent such as hexane, is disclosed in U.S. Pat. No. 6,118,020, the contents of which is incorporated herein by reference in its entirety. Acid addition salts of dodecyl 2-(N,N-dimethylamino)-propionate (DDAIP) can be inorganic as well as organic. Representative inorganic acid addition salts include the hydrochloric, hydrobromic, sulfuric, phosphoric, nitric acid addition salts of DDAIP, and their solvates. Exemplary organic acid addition salts include acetic, benzoic, salicylic, glycolic, succinic, nicotinic, tartaric, maleic, malic, palmoic, methanesulfonic, cyclohexanesulfamic, picric, and lactic acid addition salts, as well as their respective solvates. Preferred among the inorganic acid addition salts are DDAIP hydrogen chloride, and DDAIP dihydrogen sulfate.

Suitable (N-substituted amino)-alkanol alkanoates can be represented by the formula:

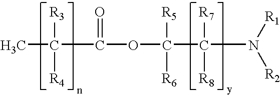

wherein n is an integer having a value in the range of about 5 to about 18; y is an integer having a value in the range of 0 to about 5; and R₁, R₂, R₃, R₄, R₅, R₆, and R₇ are members of the group consisting of hydrogen, C₁ to C₈ alkyl, and C₃ to C₈ aryl; and R₈ is a member of the group consisting of hydrogen, hydroxyl, C₁ to C₈ alkyl, and C₃ to C₈ aryl. Preferred are (N-substituted amino)-alkanol alkanoates such as C₅ to C₁₈ carboxylic acid esters and pharmaceutically acceptable salts thereof. Preferred (N-substituted amino)-alkanol alkanoates include (N,N-disubstituted amino)-alkanol alkanoates.

In general, suitable penetration enhancers can be chosen from those listed above as well as sulfoxides, alcohols, fatty acids, fatty acid esters, polyols, amides, surfactants, terpenes, alkanones, organic acids and mixtures thereof. See generally Chattaraj, S. C. and Walker, R. B., Penetration Enhancer Classification, pp. 5-20 in Maibach, H. I., and Smith, H. E., (eds.), Percutaneous Penetration Enhancers, CRC Press, Inc., Boca Raton, Fla. (1995) and Büyüktimkin, N., et al., Chemical Means of Transdermal Drug Permeation Enhancement, in Gosh, T. K., et al., (eds.) Transdermal and Topical Drug Delivery Systems, Interpharm Press, Inc., Buffalo Grove, Ill. (1997). Suitable sulfoxides include dimethylsulfoxide, decylmethylsulfoxide and mixtures thereof. Suitable alcohols include ethanol, propanol, butanol, pentanol, hexanol, octanol, nonanol, decanol, 2-butanol, 2-pentanol, benzyl alcohol, caprylic alcohol, decyl alcohol, lauryl alcohol, 2-lauryl alcohol, myristyl alcohol, cetyl alcohol, stearyl alcohol, olcyl alcohol, linolyl alcohol, linolenyl alcohol and mixtures thereof. Suitable fatty acids include valeric, heptanoic, pelargonic, caproic, capric, lauric, myristic, stearic, oleic, linoleic, linolenic, caprylic, isovaleric, neopentanoic, neoheptanoic, neononanoic, trimethyl hexanoic, neodecanoic and isostearic acids and mixtures thereof.

Suitable fatty acid esters include isopropyl n-butyrate, isopropyl n-hexanoate, isopropyl n-decanoate, isopropyl myristate, isopropyl palmitate, octyldodecyl myristate, ethyl acetate, butyl acetate, methyl acetate, methylvalerate, methylpropionate, diethyl sebacate, ethyl oleate, ethyl laurate and mixtures thereof. Suitable polyols include propylene glycol, polyethylene glycol, ethylene glycol, diethylene glycol, triethylene glycol, dipropylene glycol, glycerol, propanediol, sorbitol, dextrans, butanediol, pentanediol, hexanetriol and mixtures thereof.

Suitable amides include urea, dimethylacetamide, diethyltoluamide, dimethylformamide, dimethyloctamide, dimethyldecamide, 1-alkyl-4-imidazolin-2-one, pyrrolidone derivatives, cyclic amides, hexamethylenelauramide and its derivatives, diethanolamine, triethanolamine and mixtures thereof. Suitable pyrrolidone derivatives include 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lauryl-4-carboxy-2-pyrrolidone, 1-decyl-thioethyl-2-pyrrolidone (HP-101), 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-cyclohexylpyrrolidone, N-dimethylaminopropylpyrrolidone, N-cocoalkypyrrolidone, N-tallowalkypyrrolidone, fatty acid esters of N-(2-hydroxymethyl)-2-pyrrolidone and mixtures thereof. Suitable cyclic amides include 1-dodecylazacycloheptane-2-one (laurocapram, Azone®), 1-geranylazacycloheptan-2-one, 1-farnesylazacycloheptan-2-one, 1-geranylgeranylazacycloheptan-2-one, 1-(3,7-dimethyloctyl)azacycloheptan-2-one, 1-(3,7,11-trimethyloctyl)azacycloheptan-2-one, 1-geranylazacyclohexane-2-one, 1-geranylazacyclopentan-2,5-dione, 1-farnesylazacyclopentan-2-one and mixtures thereof.

Suitable surfactants include anionic surfactants, cationic surfactants, nonionic surfactants, bile salts and lecithin. Suitable anionic surfactants include sodium laurate, sodium lauryl sulfate and mixtures thereof. Suitable cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride, and mixtures thereof. Suitable nonionic surfactants include α-hydro-ω-hydroxy-poly(oxyethylene)-poly(oxypropyl) poly(oxyethylene)block copolymers, polyoxyethylene ethers, polyoxyethylene sorbitan esters, polyethylene glycol esters of fatty alcohols and mixtures thereof. Suitable α-hydro-ω-hydroxy-poly(oxyethylene)-poly(oxypropyl) poly(oxyethylene)block copolymers include Poloxamers 231, 182, and 184 and mixtures thereof. Suitable polyoxyethylene ethers include 4-lauryl ether (BRIJ 30™), (BRIJ 93™), (BRIJ 96™), 20-oleyl ether (BRIJ 99™) and mixtures thereof. Suitable polyoxyethylene sorbitan esters include the monolaurate (TWEEN 20™, SPAN 20™) the monopalmitate (TWEEN 40™), the monostearate (TWEEN 60™), and the monooleate (TWEEN 80™) and mixtures thereof. Suitable polyethylene glycol esters of fatty acids include the 8-oxyethylene stearate ester (MYRJ 45 ™), (MYRJ 51 ™), the 40-oxyethylene stearate ester (MYRJ 52™) and mixtures thereof. Suitable bile salts include sodium cholate, sodium salts of laurocholic, glycolic and desoxycholic acids and mixtures thereof.

Suitable terpenes include D-limonene, α-pinene, β-enrene, α-terpineol, terpinen-4-ol, carvol, carvone, pulegone, piperitone, menthone, menthol, geraniol, cyclohexene oxide, limonene oxide, α-pinene oxide, cyclopentene oxide, 1,8-cineole, ylang ylang oil, anise oil, chenopodium oil, eucalyptus oil and mixtures thereof. Suitable alkanones include N-heptane, N-octane, N-nonane, N-decane, N-undecane, N-dodecane, N-tridecane, N-tetradecane, N-hexadecane and mixtures thereof. Suitable organic acids include citric acid, succinic acid, salicylic acid, salicylates (including the methyl, ethyl and propyl glycol derivatives), tartaric acid and mixtures thereof.

Natural and modified polysaccharide gums are also an important ingredient of the composition. Suitable representative gums are those in the natural and modified galactomannan gum category. A galactomannan gum is a carbohydrate polymer containing D-galactose and D-mannose units, or other derivatives of such a polymer. There is a relatively large number of galactomannans, which vary in composition depending on their origin. The galactomannan gum is characterized by a linear structure of β-D-mannopyranosyl units linked (1→4). Single membered α-D-manopyranosyl units, linked (1→6) with the main chain, are present as side branches. Galactomannan gums include guar gum, which is the pulverized endosperm of the seed of either of two leguminous plants (Cyamposis tetragonalobus and psoraloids) and locust bean gum, which is found in the endosperm of the seeds of the carobtree (ceratonia siliqua). Suitable modified polysaccharide gums include ethers of natural or substituted polysaccharide gums, such as carboxymethyl ethers, ethylene glycol ethers and propylene glycol ethers. An exemplary substituted polysaccharide gum is methylcellulose.

Other suitable representative gums include agar gum, carrageenan gum, ghatti gum, karaya gum, rhamsan gum and xanthan gum. The composition of the present invention may contain a mixture of various gums, or mixture of gums and acidic polymers.

Gums, and galactomannan gums in particular, are well-known materials. See for instance, Industrial Gums: Polysaccharides & Their Derivatives, Whistler R. L. and BeMiller J. N. (eds.), 3rd Ed. Academic Press (1992) and Davidson R. L., Handbook of Water-Soluble Gums & Resins, McGraw-Hill, Inc., N.Y. (1980). Most gums are commercially available in various forms, commonly a powder, and ready for use in foods and topical compositions. For example, locust bean gum in powdered form is available from Tic Gums Inc. (Belcam, Md.).

When present, the polysaccharide gums are present in the range from about 0.1 percent to about 5 percent, based on the total weight of the composition, with the preferred range being from 0.5 percent to 3 percent. In one preferred embodiment, 2.5 percent by weight of a polysaccharide gum is present. Illustrative compositions are given in the examples, below.

An optional alternative to the polysaccharide gum is a polyacrylic acid polymer. A common variety of polyacrylic acid polymer is known generically as “carbomer.” Carbomer is polyacrylic acid polymers lightly cross-linked with polyalkenyl polyether. It is commercially available from the B. F. Goodrich Company (Akron, Ohio) under the designation “CARBOPOL™.” A particularly preferred variety of carbomer is that designated as “CARBOPOL 940.”

Other polyacrylic acid polymers suitable for use are those commercially available under the designations “Pemulen™” (B. F. Goodrich Company) and “POLYCARBOPHIL™” (A. H. Robbins, Richmond, Va.). The Pemulen™ polymers are copolymers of C₁₀ to C₃₀ alkyl acrylates and one or more monomers of acrylic acid, methacrylic acid or one of their simple esters crosslinked with an allyl ether of sucrose or an allyl ether of pentaerythritol. The POLYCARBOPHIL™ enhancer is a polyacrylic acid cross-linked with divinyl glycol.

Where polyacrylic acid polymers are present, they represent about 0.5 percent to about 5 percent of the composition, based on its total weight.

Another important component of the present invention is a lipophilic component. The term lipophilic component as used herein refers to an agent, preferably a mixture of agents, that is both lipophilic and hydrophilic. The C₁ to C₈, aliphatic alcohols, the C₂ to C₃₀ aliphatic esters, and their mixtures can serve as lipophilic component. Illustrative suitable alcohols are ethanol, n-propanol and isopropanol, while suitable esters are ethyl acetate, butyl acetate, ethyl laurate, methyl propionate, isopropyl palmitate and isopropyl myristate. As used herein, the term “aliphatic alcohol” includes polyols such as glycerol, propylene glycol and polyethylene glycols. A mixture of alcohol and ester is preferred, and in particular, a mixture of ethanol and ethyl laurate is most preferred. The concentration of lipophilic component required necessarily varies according to other factors such as the desired semi-solid consistency and the desired skin penetration promoting effects. The preferred topical composition contains lipophilic compound in the range of 7 percent to 40 percent by weight based on the total weight of the composition. Where a lipophilic component that is a mixture of aliphatic alcohol and aliphatic ester is used, the preferred amount of alcohol is in the range of 5 percent to 15 percent, while that of aliphatic ester is in the range from 2 percent to 15 percent (again based on the total weight of the composition). Where polyethylene glycol is used, polyethylene glycol is present in the amount of about 1 weight percent to about 25 weight percent, based on the total weight of the composition. In preferred embodiments, a liquid polyethylene glycol is used to preserve the desired semi-solid consistency of the topical composition, such as polyethylene glycol 200, polyethylene glycol 400 or polyethylene glycol 600 A preferred polyethylene glycol is polyethylene glycol 400 (PEG 400). When present, polyethylene glycol 400 is about 1 weight percent to about 25 weight percent, preferably about 3 weight percent to about 20 weight percent, based on the total weight of the composition.

In one embodiment, the C₂ to C₃₀ aliphatic esters, and their mixtures comprising the lipophilic compound include C₈ to C₃₀ aliphatic esters of glycerol selected from the group consisting monoglycerides, diglycerides, triglycerides, and mixtures thereof. Suitable aliphatic esters include glyceryl esters of saturated fatty acids, unsaturated fatty acids and mixtures thereof. Suitable saturated fatty acids include caproic acid, caprylic acid, capric acid, lauric acid, myristic acid, palmitic acid, stearic acid, arachidic acid, behenic acid and lignoceric acid. Suitable unsaturated fatty acids include oleic acid, linoleic acid and linolenic acid. Suitable glyceryl esters include glyceryl monooleate, triolein, trimyristin and tristearin, preferably trimyristin.

The concentration of lipophilic compound required necessarily varies according to other factors such as the desired semi-solid consistency and the desired skin penetration promoting effects. Suitably the concentration of lipophilic compound is in the range of 0.5 percent to 40 percent by weight based on the total weight of the composition. The preferred topical composition contains lipophilic compound in the range of 7 percent to 40 percent by weight based on the total weight of the composition.

Where a mixture of aliphatic alcohol and aliphatic ester are employed, the suitable amount of alcohol is in the range of about 0.5 percent to 10 percent. In one preferred embodiment, the amount of alcohol is in the range of about 5 percent to 15 percent, while that of aliphatic ester is in the range of about 2 percent to 15 percent (again based on the total weight of the composition). In another preferred embodiment, the amount of alcohol is in the range of about 0.5 percent to 10 percent, while that of aliphatic ester is in the range of 0 percent to 10 percent (again based on the total weight of the composition).

An optional, but preferred, component of the present invention is an emulsifier. Although not a critical factor, preferable emulsifiers generally exhibit a hydrophilic-lipophilic balance (HLB) number of at least 9. Sucrose esters, and specifically sucrose stearate, can serve as emulsifiers for the topical composition of the present invention. Sucrose stearate is a well known emulsifier available from various commercial sources.

Typical non-ionic surfactants include the polysorbates, which are mixtures of partial esters of sorbitol and its mono- and dianhydrides, typically condensed with approximately 20 mol of ethylene oxide; polyethyoxylated alkyl ethers and esters, in which the alkyl chain can be either saturated, unsaturated, branched or linear; polyethoxylated alkyl phenols, in which the hydrophobic group normally octyl or nonylphenol; and poloxamers, polyoxyethylene-polyoxypropylene block copolymers, in which the polyoxypropylene chain acts as the hydrophobic moiety. Some commercially available non-ionic surfactants are BRIJ 99™, BRIJ 78™, polyoxyl 40 stearate and polysorbate 80. BRIJ 99™ and BRIJ 78™ are polyethylene glycol fatty alcohol ethers. Polyoxyl 40 stearate is a mixture of mono- and distearate esters of polyoxyethylene and of free polyoxyethylene. Polysorbate 80 is polyoxyethylene (20) sorbitan monooleate.

When an emulsifier is used, sucrose stearate present up to about 2 percent, based on the total weight of the composition, is preferred. The preferred amount of sucrose stearate emulsifier can also be expressed as a weight ratio of emulsifier to polysaccharide gum. A ratio of 1 to 6 emulsifier to gum is preferred generate the desired semi-solid consistency and separation resistance.

The present invention includes a buffer system. Buffer systems serve to maintain or buffer the pH of compositions within a desired range. The term “buffer system” or “buffer” as used herein has reference to a solute agent or agents which, when in a water solution, stabilize such solution against a major change in pH (or hydrogen ion concentration or activity) when acids or bases are added thereto. Solute agent or agents which are thus responsible for a resistance to change in pH from a starting buffered pH value in the range indicated above are well known. While there are numerous other suitable buffers, such as acetate buffers, potassium phosphate monohydrate has proven effective for compositions of the present invention.

The final pH value of the pharmaceutical composition of the present invention may vary within the physiologically compatible range. Necessarily, the final pH value is not irritating to human skin. Without violating this constraint, the pH may be selected to improve prostaglandin E₁, stability and to adjust consistency when required. With these factors accounted for, the preferred pH value is about 3.0 to 7.4. The most preferred pH range is from about 3.5 to about 6.0.

The remaining component of the composition is water, which is preferably purified. The composition contains water in the range of about 50 to about 90 percent, optionally including at least some of the water in the buffer, based on the total weight of the composition. The specific amount of water present is not critical, however, being adjustable to obtain the desired consistency and/or concentration of the other components.

Additionally, known transdermal penetration enhancers can also be added, if desired. Illustrative are dimethyl sulfoxide (DMSO), dimethyl acetamide (DMA), 2-pyrrolidone, N,N-diethyl-m-toluamide (DEET), 1-dodecylazacycloheptane-2-one (laurocapram, Azone®, a registered trademark of Nelson Research), N,N-dimethylformamide, N-methyl-2-pyrrolidone, calcium thioglycolate, oxazolidinones, alkyl-2-(N-substituted amino) alkanoates and their acid addition salts (e.g., dodecyl N,N-dimethylamino isoproprionate (DDAIP)), N-substituted amino alkanol alkanoates and their acid addition salts, dioxolane derivatives, laurocapram derivatives, macrocyclic enhancers such as macrocyclic ketones and mixtures thereof.

Stabilizers, coloring agents, rheological agents, fragrances and preservatives can be added to the extent that they do not overly limit prostaglandin E₁ skin penetration or prevent the desired semi-solid consistency. When present, such are usually added in amounts of about 0.05 to about 0.30%. Suitable preservatives include methylparabens (methyl PABA), propylparabens (propyl PABA) and butylhydroxy toluene (BHT).

The compositions of the present invention can also include a small amount of a topical anesthetic, if desired. When present, the topical anesthetic comprises about 0.01 to about 20 percent by weight, preferably about 0.01 to about 10 percent by weight based on the weight of the composition. As can be recognized, the suitable concentration of topical anesthetic will vary, depending the specific anesthetic and the presence of other components. For example, suitable concentrations include about 1 to about 20 percent by weight of benzocaine, about 0.25 to about 2.5 percent by weight of dibucaine, about 0.01 to about 10 percent by weight of lidocaine, or about 0.25 to about 1 percent by weight of tetracaine.

In preferred embodiments, the topical composition comprises at least one local anesthetic. Suitable local anesthetics include those approved for topical application, including, but not limited to ambucaine, amolanone, amylocalne hydrochloride, articaine, benoxinate, benzocaine, betoxycaine, biphenamine, bupivacaine, butacaine, butamben, butanilicaine, butethamine, butoxycaine, carticaine, chloroprocaine hydrochloride, cocaethylene, cocaine, cyclomethycaine, dibucaine hydrochloride, dimethocaine, diperodon hydrochloride, dyclonine, ecgonidine, ecgonine, ethyl chloride, etidocaine, beta-eucaine, euprocin, fenalcomine, fomocaine, hexylcaine hydrochloride, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octacaine, orthocaine, oxethazaine, parethoxycaine, phenacaine hydrochloride, phenol, piperocaine, piridocaine, polidocanol, pramoxine, prilocalne, procaine, propanocaine, proparacaine, propipocaine, propoxycaine hydrochloride, pseudococaine, pyrrocaine, ropivacaine, salicyl alcohol, tetracaine hydrochloride, tolycaine, trimecaine, zolamine and mixtures thereof.

In general, with few exceptions, useful local anesthetics contain a lipophilic radical (mostly of aromatic structure), an intermediate chain and a hydrophilic radical (often an amino group). Local anesthetics can be further classified chemically as alcohols and alkyl ethers (such as chlorbutanol, benzyl alcohol, saligenine and pistocaine), amines, amino alcohols, amino alkyl ethers (such as pramocaine and dimethisoquine), amino ketones (such as falicaine), carboxylic acid esters (such as benzocaine, procaine and parophoxycaine), carboxylic acid amides (such as lidocaine and dibucaine), carbamic acid esters (such as diperodone) and amidines and guanidines (such as phenacaine and guanicaine). See Büchi, J., and Perlia, X., “Structure—Activity Relations and Physico-Chemical Properties of Local Anesthetics. Part I. Relations between Chemical Structure and Local Anesthetic Activity,” pp. 39-130 in Int. Encycl. Pharm. Therapeut. Local Anesthetics, Vol. I, Pergamon Press, New York, 1971.

In preferred embodiments, the local anesthetic molecular structure consists of a tertiary amine linked to a substituted aromatic ring by an intermediate chain. In some embodiments, the intermediate chain includes both a carbonyl group and one or more alkyl groups. The intermediate chain may further contain an ester linkage or an amide linkage. Suitable aminoamide local anesthetics include lidocaine, bupivacaine, mepivacaine, dibucaine, propivacaine, etidocaine and tocainide. Suitable aminoester local anesthetics include procaine, chloroprocaine, tetracaine, isocaine, benzocaine, and monocaine. In embodiments in which the intermediate chain includes both a carbonyl group and one or more alkyl groups, a preferred local anesthetic is dyclonine, 1-(4-butoxyphenyl)-3-(1-piperidynyl)-1-propanone.

Preferred local anesthetics are those producing a moderate duration of anesthesia, more preferably those having a long duration of anesthetic action. For example, procaine and chloroprocaine have a short duration of action. Lidocaine, mepivacaine and prilocalne produce a moderate duration of anesthesia. Suitable long-acting local anesthetics include ropivacaine, tetracaine, bupivacaine and etidocaine.

Contemplated dosage forms of the semi-solid pharmaceutical composition of the present invention are creams, gels, and the like, also including but not limited to compositions suitable for use with transdermal patches and like devices.

The semi-solid composition of the present invention has a suitably chosen viscosity such that the composition is naturally retained where applied. The semi-solid composition can exhibit Newtonian or non-Newtonian rheological characteristics. In some preferred embodiments, the semi-solid composition of the present invention exhibits non-Newtonian rheological characteristics, i.e. in which the apparent viscosity is dependent on the shear rate applied to the composition. Preferably the composition has “shear-thinning” rheological properties. As used herein, “shear-thinning” refers to a reduction in apparent viscosity (the ratio of shear stress to the shear rate) with increasing shear rate, whether the reduction in apparent viscosity is time independent (pseudoplastic), time dependent (thixotropic) or associated with a yield stress, defined as a stress that must be exceeded before flow starts, (Bingham plastics and generalized Bingham plastics). See, generally, Harris, J., & Wilkinson, W. L., “Non-newtonian Fluid,” pp. 856-858 in Parker, S. P., ed., McGraw-Hill Encyclopedia of Physics, Second Edition, McGraw-Hill, New York, 1993. Suitable viscosity ranges from about 5,000 centipoise (cps) to about 20,000 cps, preferably from about 7,000 cps to about 13,000 cps.

The topical composition is applied about five to about twenty minutes before sexual intercourse to the labia, clitoris and vagina and massaged until absorption is complete. In a preferred embodiment of the present invention, the topical composition is applied about five to about twenty minutes before sexual intercourse to the clitoris and the vaginal Grafenberg spot (also known as Grafenberg zone or G-spot), a region on the inner anterior wall of the vagina about 5 cm internal to the vaginal opening. In other preferred embodiments, the composition is applied to the inner vaginal wall, including the G-spot, but not to the clitoris.

Amounts of the topical composition ranging between about 0.1 and about 10 grams, preferably about 0.1 to about 3 grams are sufficient for vasodilation and the beneficial effects to occur. The present invention can be used with or without benefit of erotic stimuli. The determination of an ideal dose of the composition should be determined with each individual by one skilled in the art, such as a physician or sex therapist. The effective amount to be administered is selected to provide increased blood flow to the genitalia, which may be assessed by visual inspection, vaginal photoplethysmography, vaginal lubrication or engorgement. The preferred active component is prostaglandin, most preferably prostaglandin E₁. Suitable doses of these selected drugs and other suitable drugs, such as phentolamine will be apparent to those skilled in the art, or may be deduced from the literature in combination with the teaching of the present disclosure.

While this invention has been described by way of preferred embodiments, the examples set out herein are not intended to limit the scope of the invention which contemplates the use of any pharmacologic vasodilating drug capable of absorption into the local and systemic circulation upon administration of the drug via the transmucosal, transdermal, intranasal, buccal or rectal routes of administration.

Numerous other advantages of the present invention will be apparent from the following detailed description of the invention including the accompanying examples and the appended claims.

### EXAMPLE 1

### Formulation of Suitable Compositions

Composition I was prepared as follows according to Formulation I (Table 1, below). Part A was formed by dissolving about 0.4 parts prostaglandin E₁ (Alprostadil USP) in about 5 parts ethyl alcohol. Next, about 5 parts ethyl laurate were mixed into the alcohol-prostaglandin E₁ solution. Part B was prepared starting from a pH 5.5 water/buffer solution. The water/buffer solution was prepared by adding sufficient potassium phosphate monobasic to purified water to create a 0.1 M solution. The water/buffer solution diluted to a final concentration of about 0.05M and about pH 5.5, adjusted with a strong base solution (1 N sodium hydroxide) and a strong acid (1 N phosphoric acid). Suitable buffer concentrations range from about 0.005M to about 1.0M. Preferred buffer concentrations range from about 0.05M to about 0.2M. In several preferred embodiments the buffer concentration is 0.1M. Propylene glycol (about 5 parts) was added to the water/buffer solution, and then the polyacrylic polymer (about 1 part) was dispersed in the propylene glycol/water/buffer solution. All parts specified herein are parts by weight.

Parts A and B were mixed and homogenized using a homogenizer. Table 1, below, contains a list of ingredients and proportions. The resulting composition was a spreadable, semi-solid suitable for application to the skin and mucous membranes without the need for supporting devices such as patches and adhesive strips. The composition was both homogenous in appearance and resistant to separation. Compositions based on formulations II-VII were prepared following the same procedure.

TABLE 1

| | Formulation (weight %) | | | | | | |
|---|---|---|---|---|---|---|---|
| Component | I | II | III | IV | V | VI | VII |
| Noveon AA-1 | 1 | 1 | 1 | 1 | | | |
| Ethanol | 5 | 5 | | 5 | 5 | 5 | 5 |
| Propylene | 5 | 5 | 5 | | | | |
| glycol | | | | | | | |
| Ethyl laurate | 5 | 5 | | 5 | 5 | 3 | 3 |
| 70% Sorbitol | | 5 | | | | | |
| Glycerol | | | 5 | | | | |
| DDAIP | | | | 5 | | 2.5 | |
| DDAIP HCl | | | | | | | 2.5 |
| Sesame oil | | | 5 | | | | |
| Squalene | | | 5 | | | | |
| Prehydrated | | | | | 3 | | |
| Locust | | | | | | | |
| bean gum | | | | | | | |
| Modified Guar | | | | | | 3 | 2.5 |
| Gum | | | | | | | |
| Sucrose | | | | | 0.5 | | |
| stearate | | | | | | | |
| 0.05M pH 5.5 | 78.85 | 73.85 | 73.85 | 78.85 | 86.1 | 81 | |
| buffer | | | | | | | |
| 0.1M pH 5.5 | | | | | | | 87 |
| buffer | | | | | | | |
| 1M NaOH | 4.75 | 4.75 | 4.75 | 4.75 | | | |
| Prostaglandin | 0.4 | 0.4 | 0.4 | 0.4 | 0.4 | 0.3 | 0.3 |
| E₁ | | | | | | | |

As noted above, in other embodiments, such as Compositions VI and VII, the composition may include a modified polysaccharide gum, suitably a modified galactomannan gum, such as a guar gum. Alternatively, a polyacrylic polymer may be used instead of the polysaccharide gum.

### EXAMPLE 2

### In Vitro Penetration of Different Formulations

The relative ability of compositions prepared according to the formulations of Table 1 to provide prostaglandin E₁ was studied in two in vitro model systems corresponding to skin and mucosal membranes: shed snake skin and sheep vaginal membrane.

Compositions were evaluated for skin penetration using shed snake skin as a model barrier. Shed snakeskin was obtained from the Animal Care Unit of the University of Kansas. With head and tail sections removed, the skin was randomly divided into test sections and then hydrated by soaking.

Samples of the compositions listed in Table 1 were evaluated using modified Franz-type diffusion cells (surface area 1.8 cm²). Specifically, skin pieces were mounted on top of a receptor cell of a vertical diffusion cell assembly in which a small magnetic bar was inserted and filled with an isotonic buffer. A seal was placed on top of the skin section followed by a donor cell. The two cells were then clamped together. Known amounts of the formulations were applied on the bottom of a small capped vial (weight about 5 grams) which fits exactly to the donor cell to ensure uniform distribution. The vials were placed on the skin in the donor cell. To reduce the evaporation of the ingredients, the donor cell and vial were gently taped together with a water-resistant adhesive band. The cells were transferred to a stirred water bath that was maintained at 37 degrees Celsius. Samples were withdrawn from the cells each hour for four hours and analyzed for the concentration of prostaglandin E₁, with changes in concentration indicating the amount penetrating. Tests with multiple skin samples from the same snake yielded data that were averaged.

For a discussion of the use of shed snake skin in the evaluation of drug penetration, see U.S. Pat. No. 4,771,004 to Higuchi, which is incorporated herein by reference.

The results of the penetration study are presented in Table 2, below. Prostaglandin E₁ penetrated quickly at a relatively sustained rate for four hours from compositions prepared based on Formulations I, II and III. In contrast, relatively little penetration was observed using compositions based on Formulations IV and V.

TABLE 2
Prostaglandin E₁ Average Cumulative Amount (μg/cm²)

| | | | | | Formu- |
|---|---|---|---|---|---|
| | Formulation | Formulation | Formulation | Formulation | lation |
| Hour | I | II | III | IV | V |
| 1 | 5.00 | 2.89 | 1.58 | 3.55 | 0.39 |
| 2 | 8.42 | 6.32 | 2.11 | 8.42 | 0.92 |
| 3 | 12.37 | 11.58 | 2.11 | 16.58 | 2.11 |
| 4 | 18.68 | 17.11 | 1.58 | 23.82 | 4.21 |

### EXAMPLE 3

### Concentration Effects on In Vitro Penetration

The effect of the prostaglandin E₁ concentration on permeation was studied using stripped shed snake skin. Stripped shed snake skin was prepared by removing the outer scale layer of the shed snake skin by 3-5 cycles of application and removal of adhesive tape (Minnesota Mining and Manufacturing Co., St. Paul, Minn.). The compositions tested were prepared as described in Example 1, and had final proportions (parts) of prostaglandin E₁, (either 0.05%, 0.1%, or 0.2%); ethanol, about 5 parts; propylene glycol, about 5 parts; ethyl laurate, about 5 parts; polyacrylic polymer, about 1 part; 1M NaOH about 4.75 parts; 0.005M phosphate buffer, about pH 5.5, q.s. 100.

Penetration studies were performed as described in Example 2. The results are shown in Table 3, below. Higher prostaglandin E₁ concentrations produce both more rapid permeation and a higher amount delivered.

TABLE 3
Prostaglandin E₁
Cumulative Amount (μg/cm²)

| | 0.05% Prostaglandin | 0.1% Prostaglandin | 0.2% Prostaglandin |
|---|---|---|---|
| | E₁ | E₁ | E₁ |
| Hour | (Open Squares) | (Filled Triangles) | (Filled Squares) |
| 1 | 25 | 33.75 | 41 |
| 2 | 40 | 65 | 81 |
| 3 | 50 | 85 | 118 |
| 4 | 58.75 | 102.5 | 143 |

### EXAMPLE 4

### Comparison of Permeation in Two Model Membrane Systems

The permeation of prostaglandin E₁ in a topical composition of the present invention was compared using the stripped shed snake skin of Example 3 and the sheep vaginal membrane in vitro system. The topical composition used was the 0.2% prostaglandin E₁ composition of Example 3.

Sheep vaginas were obtained from a local slaughterhouse. The freshly excised organ was refrigerated and used immediately. After excision, the outer wall of the vagina was carefully separated from any adhering tissue, taking care to avoid damage. The vagina was cut open longitudinally (vertically) (Kabadi, M. B., and Chien, Y. W., Intravaginal controlled administration of Fluorogestone acetate. II: Development of an in vitro system for studying the intravaginal release and permeation of Fluorogestone acetate, J. Pharm. Sci. 73: 1464-1468 (1984)). The vaginal mucosa were separated from the interior of the vaginal wall, soaked in nanopure water, cut into appropriately sized pieces, and mounted in using modified Franz-type diffusion cells as described in Example 2.

Penetration studies were performed as described in Example 2. The results are shown in Table 4, below. The penetration measured in stripped shed snake is comparable to that measured in sheep vaginal membrane over the first two hours, diverging slightly at three hours.

TABLE 4
Prostaglandin E₁
Average Cumulative Amount (μg/cm²)

| | Stripped Shed Snake Skin | Sheep Vaginal Membrane |
|---|---|---|
| Hour | (Membrane I) | (Membrane II) |
| 1 | 11.67 | 8.33 |
| 2 | 30.00 | 32.92 |
| 3 | 50.00 | 60.00 |
| 4 | 55.83 | 85.42 |

### EXAMPLE 5

Clinical Study in Women Suffering from Sexual Dysfunction

This study was conducted to evaluate the efficacy and safety of placebo and 3 doses of topical prostaglandin E₁ cream (compositions based on Formulation I of Example 1 containing either 0.05%, 0.1% or 0.2% prostaglandin E₁) in female subjects with FSAD in a controlled laboratory setting. Efficacy was assessed by vaginal photoplethysmography (Geer Gauge) during visual sexual stimulation (VSS), and by the use of quantitative patient questionnaires and diaries. Premenopausal subjects were enrolled under the assumption that their inherent magnitude of physiologic response is greater than that of postmenopausal subjects and thus will improve the likelihood of measuring pharmacologic effects. The study assessed the safety of 3 doses of topical prostaglandin E₁ cream in women with sexual dysfunction. The study also assessed the efficacy of 3 doses of topical prostaglandin E₁ cream in affecting vaginal blood flow and exudates and refinement of a quality of life instrument.

The study was a single center, single-blind, escalating dose, placebo-controlled pilot study to investigate the physiologic action, and the efficacy and safety of 3 doses of topical prostaglandin E₁ cream in women with FSAD. A total of 8 subjects were enrolled in this study. The study measured the dose-response characteristics of the safety and efficacy of the prostaglandin E₁ cream in terms of physiological response as well as the subjects' signs and symptoms and perceptions of the physiologic responses.

After signing the informed consent, at screening (Visit 1), the subjects underwent an adaptation session in the sexual response assessment laboratory. This session was intended to allow the patient to become familiar and comfortable with the procedures that will be carried out at subsequent visits. A complete medical history (including the Sexual Activity Questionnaire and the Brief Index of Sexual Function for Women (BISF-W; Taylor, J. F., et al., Self-report assessment of female sexual function: psychometric evaluation of the Brief Index of Sexual Functioning for Women. Arch Sexual Behavior: 23: 627-643, 1994.)) were collected. All medications taken by the patient were be recorded. Baseline safety assessments included ECG, physical exam (including pelvic exam), clinical laboratory tests, and vital signs. Subjects who met all inclusion and no exclusion criteria continued in the study.

The inclusion criteria for the study were that the female subjects aged 21 and provided written, informed consent; had a history of female sexual dysfunction (defined as impairment of the woman's ability to experience vaginal lubrication or engorgement sufficient for intercourse on at least 50% of attempts) of at least 6 months duration; were premenopausal; used adequate contraception (oral hormonal contraceptives, hormonal implants, or tubal ligation); had regular menses (cycles consistent in duration±2 days and between 25 and 31 days in length; and had a normal Pap smear within the past year. Any patient with an ASCUS Pap, except, “ASCUS, favor dysplasia” was admitted. Any Pap smear with inflammation or inflammatory changes in the absence of clinically significant vaginitis was admitted.

Subjects with any of the following conditions or meeting any of the following criteria were excluded from the study: female sexual dysfunction caused by untreated endocrine disease, e.g., hypopituitarism, hypothyroidism, diabetes mellitus; positive serum beta HCG or UPT result; a history of chronic or complicated urinary tract or vaginal infections within previous 12 months; a history of pelvic inflammatory disease within previous 12 months; history of dyspareunia not attributable to vaginal dryness within previous 12 months; significant (moderate to severe) vaginal atrophy; presence of moderate to severe vaginitis on pelvic examination; cervical dysplasia; significant cervicitis as manifested by mucopurulent discharge from the cervix; evidence of clinically significant hepatic disease as evidenced by SGOT or SGPT>3 times the upper limit of normal within the last 6 months; evidence of clinically significant renal disease as evidenced by a serum creatinine>2.5 mg % within the last 6 months; a history of myocardial infarction within previous 12 months; symptomatic coronary artery disease, i.e., angina pectoris; symptomatic hypotension requiring medical consultation within the last 6 months; psychoses, uncontrolled bipolar disorder, uncontrolled depression; acute or chronic disease requiring frequent changes (changes within previous two months or anticipated in following two months) in medications or doses of chronic therapy; significant central nervous system diseases within the last 6 months i.e., stroke, spinal cord injury, etc.; participation in another study with an investigational drug or device during the 30 days prior to study entry, or planned during the study; any condition which would interfere with the patient's ability to provide informed consent, to comply with study instructions, or which might confound the interpretation of the study results; or any condition which would endanger the participant if she participated in this trial.

At Visit 2, subjects received a single-blinded intravaginal dose of placebo. The patient underwent visual sexual stimulation in the sexual response assessment laboratory. The Geer Gauge (vaginal photoplethysmograph) was applied according to the manufacturer's instructions and vaginal photoplethysmography was recorded continuously from 15 minutes prior to dosing until the end of the visual sexual stimulation, approximately 60 minutes postdose. Safety was assessed from vital sign measurements and by monitoring the occurrence of adverse events. The patient's external genitalia, the vagina, and the cervix were inspected. Questionnaires were administered.

At Visit 3, subjects received a single-blinded dose of prostaglandin E₁ cream (containing 0.5 mg PGE₁) applied to the labia, clitoris and the vulvar region of the vagina. All procedures from Visit 2 were repeated. If the patient tolerated this dose, she continued to the next visit. If the patient did not tolerate the dose in the clinic, she would dismissed from the study but receive appropriate follow-up medical care.

At Visit 4, subjects returned to the clinic and received a single-blinded dose of prostaglandin E₁ cream (containing 1.0 mg PGE₁) applied to the labia, clitoris and the vulvar region of the vagina. All procedures from Visit 2 were repeated. As before, if the patient tolerated this dose, she continued to the next visit. If the patient did not tolerate the dose in the clinic, she would dismissed from the study but receive appropriate follow-up medical care.

At Visit 5, subjects returned to the clinic and received a single-blinded dose of prostaglandin E₁ cream (containing 2.0 mg PGE₁) applied to the labia, clitoris and the vulvar region of the vagina. All procedures from Visit 2 were repeated. As before, if the patient tolerated this dose, she continued to the next visit. If the patient did not tolerate the dose in the clinic, she would dismissed from the study but receive appropriate follow-up medical care.

The eight subjects had an average age of 40.4±7.7 years, an average weight of 150.3±42.3 pounds, and an average height of 63.9±2.5 inches. There were six Caucasian, one black and one Asian. All 8 subjects completed the study. The results are presented in Tables 4-14.

Vaginal blood flow measurement, as maximum amplitude change in photoplethysmography measurements, did not show a statistically significant increase as shown in Table 5. However, there were no decreases seen on treatment. The lack of statistical significance may be related to the high baseline levels.

TABLE 5
Vaginal Blood Flow Measurement
Maximum Amplitude Change In Photoplethysmography Measurement
Meansfor Placebo, Treatment, and Within-Subject Change from
Placebo

| | PLACEBO | PGE₁ 0.05% | PGE₁ 0.1% | PGE₁ 0.2% |
|---|---|---|---|---|
| | (Visit 2) | (Visit 3) | (Visit 4) | (Visit 5) |
| | N = 6 | N = 8 | N = 8 | N = 7 |
| Mean at | 10.00 ± 4.6* | 8.50 ± 4.1 | 10.88 ± 4.6 | 5.57 ± 3.5 |
| Visit | | | | |
| Change | | −1.50 ± 2.4** | 0.50 ± 1.6 | −1.50 ± 2.4 |
| from | | (p = 0.56) | (p = 0.77) | (p = 0.56) |
| Placebo | | | | |

*Standard deviation
**Standard error

[image]

The visual inspection by the investigator revealed several significant increases in objective measures related to the treatment with the topical composition with some analyses (Table 6), in particular an increase in erythema at all dosage levels and increases in exudates at the two higher dosages at each visit. In other analyses (Tables 7, 8), the changes seen did not reach the p=0.05 criterion of significance, with the exception of the exudate observations using 0.05% PGE₁ as reference.

TABLE 6
VISUAL INSPECTION
Pre-Treatment and Post-TreatmentMeans at Each Visit

| | PLACEBO | PGE₁ 0.05% | PGE₁ 0.1% | PGE₁ 0.2% |
|---|---|---|---|---|
| | (Visit 2) | (Visit 3) | (Visit 4) | (Visit 5) |
| | N = 8 | N = 8 | N = 8 | N = 7 |
| ERYTHEMA | | | | |
| Pre | 1.00 ± 0** | 1.00 ± 0 | 1.13 ± .35 | 1.00 ± 0 |
| Post | 1.25 ± .46 | 1.63 ± .52 | 2.13 ± .83 | 2.29 ± .76 |
| Pre-Post* | p = 0.17 | p = 0.01 | p = 0.02 | p = 0.004 |
| SWELLING | | | | |
| Pre | 1.00 ± 0 | 1.00 ± 0 | 1.00 ± 0 | 1.00 ± 0 |
| Post | 1.13 ± .35 | 1.50 ± .53 | 1.25 ± .46 | 1.29 ± .49 |
| Pre-Post | p = 0.35 | p = 0.03 | p = 0.17 | p = 0.17 |
| EXUDATES | | | | |
| Pre | 1.38 ± 0.52 | 1.88 ± .64 | 1.63 ± .52 | 1.50 ± .53 |
| Post | 2.00 ± 0.76 | 2.25 ± .46 | 2.63 ± .52 | 2.57 ± .98 |
| Pre-Post | p = 0.05 | p = 0.08 | p = 0.001 | p = 0.005 |

*Change from pre-treatment to post-treatment, p-values based on paired t-tests.
**Standard deviation

TABLE 7
VISUAL INSPECTION
Comparisons of Mean Change From Pre- to Post-Treatment Within Subjects,
Using Placebo as the Reference

| | PLACEBO | PGE₁ 0.05% | PGE₁ 0.1% | PGE₁ 0.2% |
|---|---|---|---|---|
| | (Visit 2) | (Visit 3) | (Visit 4) | (Visit 5) |
| | N = 8 | N = 8 | N = 8 | N = 7 |
| ERYTHEMA* | 0.25 ± .16*** | 0.63 ± .18 | 1.00 ± .33 | 1.29 ± .29 |
| Change** | | 0.38 ± .26 | 0.75 ± .37 | 1.00 ± .31 |
| | | (p = 0.20) | (p = 0.08) | (p = 0.018) |
| SWELLING | 0.13 ± .13 | 0.50 ± .19 | 0.25 ± .16 | 0.29 ± .18 |
| Change | | 0.38 ± .26 | 0.13 ± .23 | 0.14 ± .26 |
| | | (p = 0.20) | (0.60) | (0.60) |
| EXUDATES | 0.63 ± .26 | 0.38 ± .18 | 1.00 ± .19 | 1.14 ± .26 |
| Change | | −0.25 ± .31 | 0.38 ± .26 | 0.57 ± .43 |
| | | (p = 0.45) | (p = 0.20) | (0.23) |

*Mean of the difference between pre-treatment and post-treatment, using paired t-tests.
**Mean of the pre- to post-treatment change at each active drug visit, correcting for the change during treatment with placebo.
***Standard error

TABLE 8
VISUAL INSPECTION
Comparisons of Mean Change From Pre- to Post-Treatment
Within Subjects, Using PGE₁ 0.05% as the Reference

| | PGE₁ 0.05% | PGE₁ 0.1% | PGE₁ 0.2% |
|---|---|---|---|
| | (Visit 3) | (Visit 4) | (Visit 5) |
| | N = 8 | N = 8 | N = 7 |
| ERYTHEMA* | 0.63 ± .18*** | 1.00 ± .33 | 1.29 ± .29 |
| Change** | | 0.38 ± .32 | 0.71 ± .29 |
| | | (p = 0.28) | (p = 0.047) |
| SWELLING | 0.50 ± .19 | 0.25 ± .16 | 0.29 ± .18 |
| Change | | −0.25 ± .25 | −0.14 ± .14 |
| | | (p = 0.35) | (p = 0.36) |
| EXUDATES | 0.38 ± .18 | 1.00 ± .19 | 1.14 ± .26 |
| Change | | 0.63 ± .18 | 0.71 ± .29 |
| | | (p = 0.011) | (p = 0.047) |

*Mean of the difference between pre-treatment and post-treatment, using paired t-tests.
**Mean of the pre- to post-treatment change at each active drug visit, correcting for the change during treatment with PGE 0.05%.
***Standard error

As with the vaginal blood flow measurements, the responses to the video assessment questionnaire showed high baseline responses (e.g., Table 9). The differences in responses between the lowest dose of PGE and the two higher doses are significant for question 3 (related to lubrication (Table 11) and engorgement at the highest dose level (Q4, Table 11). The graphs of the maximum responses show increased responses relative to baseline in all but one subject. The responses to questions 2 (subjective arousal) and 6 (pleasurable feelings) are near the p=0.05 level if the 0.05% and 0.2% dose levels are compared (Table 11).

TABLE 9
VIDEO ASSESSMENT QUESTIONNAIRE RESULTS
Visit Means

| | Q2 | Q3 | Q4 | Q5 | Q6 | Q9 |
|---|---|---|---|---|---|---|
| Placebo | 4.13 ± 1.6* | 4.25 ± 2.3 | 2.13 ± 2.0 | 2.00 ± 2.2 | 4.25 ± 1.9 | 6.50 ± 2.7 |
| (N = 8) | | | | | | |
| PGE₁ 0.05% | 2.25 ± 1.9 | 2.25 ± 1.7 | 1.38 ± 2.2 | 2.00 ± 2.3 | 2.75 ± 2.1 | 7.50 ± 1.5 |
| (N = 8) | | | | | | |
| PGE₁ 0.1% | 4.13 ± 1.7 | 3.88 ± 1.9 | 2.88 ± 2.7 | 2.38 ± 2.3 | 3.88 ± 2.1 | 7.75 ± 1.8 |
| (N = 8) | | | | | | |
| PGE₁ 0.2% | 4.57 ± 1.7 | 4.43 ± 2.5 | 3.71 ± 2.4 | 3.29 ± 1.8 | 5.00 ± 1.9 | 8.00 ± 2.2 |
| (N = 7) | | | | | | |

*Standard deviation

TABLE 10
VIDEO ASSESSMENT QUESTIONNAIRE RESULTS
Means of Within-Subject Differences Between Placebo and Treatment

| | Q2 | Q3 | Q4 | Q5 | Q6 | Q9 |
|---|---|---|---|---|---|---|
| Placebo vs. PGE₁ | −1.88 ± 1.1* | −2.00 ± 1.2 | −0.75 ± 1.1 | 0 ± 1.2 | −1.50 ± 1.3 | 1.00 ± 0.62 |
| 0.05% (N = 8) | (p = 0.13) | (p = 0.13) | (p = 0.52) | (p = 1.0) | (p = 0.27) | (p = 0.15) |
| Placebo vs. PGE₁ | 0 ± 0.8 | −0.38 ± 1.0 | 0.75 ± 0.5 | 0.38 ± 0.8 | −0.38 ± 1.0 | 1.25 ± 1.0 |
| 0.1% (N = 8) | (p = 1.0) | (p = 0.73) | (p = 0.20) | (p = 0.64) | (p = 0.73) | (p = 0.27) |
| Placebo vs. PGE₁ | 0.29 ± 0.4 | 0 ± 0.5 | 1.57 ± 1.0 | 1.14 ± 0.5 | 0.57 ± 0.5 | 1.00 ± 1.0 |
| 0.2% (N = 7) | (p = 0.46) | (p = 1.0) | (p = 0.17) | (p = 0.07) | (p = 0.32) | (p = 0.38) |

*Standard error

TABLE 11
VIDEO ASSESSMENT QUESTIONNAIRE
Means of Within-Subject Differences Between PGE₁ 0.05% and Treatment

| | Q2 | Q3 | Q4 | Q5 | Q6 | Q9 |
|---|---|---|---|---|---|---|
| PGE₁ 0.05% vs. | 1.88 ± 0.8* | 1.63 ± 0.7 | 1.50 ± 1.0 | 0.38 ± 0.7 | 1.13 ± 0.8 | 0.25 ± 0.6 |
| PGE₁ 0.1% (N = 8) | (p = 0.054) | (p = .04) | (p = 0.18) | (p = 0.62) | (p = 0.22) | (p = 0.68) |
| PGE₁ 0.05% vs. | 2.57 ± 1.1 | 2.57 ± 1.0 | 3.00 ± 1.2 | 1.71 ± 1.0 | 2.57 ± 1.2 | 0.29 ± 0.6 |
| PGE₁ 0.2% (N = 7) | (p = 0.06) | (p = .04) | (p = .04) | (p = 0.14) | (p = 0.08) | (p = 0.65) |

*Standard error

TABLE 12
VITAL SIGNS Means at Admission and Discharge at Each Visit

| | PLACEBO | PGE₁ 0.05% | PGE₁ 0.1% | PGE₁ 0.2% |
|---|---|---|---|---|
| | (Visit 2) | (Visit 3) | (Visit 4) | (Visit 5) |
| | N = 8 | N = 8 | N = 8 | N = 7 |
| Standing | | | | |
| Systolic | 108.8 ± 11.5* | 107.5 ± 12.6 | 103.0 ± 5.0 | 106.5 ± 7.8 |
| Admission | | | | |
| Discharge | 110.0 ± 11.3 | 103.5 ± 11.5 | 103.5 ± 11.4 | 104.0 ± 10.6 |
| Diastolic | | | | |
| Admission | 70.8 ± 5.8 | 70.8 ± 6.9 | 69.5 ± 7.2 | 71.0 ± 5.1 |
| Discharge | 72.3 ± 6.5 | 66.0 ± 7.3 | 68.8 ± 4.4 | 68.6 ± 4.1 |
| Pulse | | | | |
| Admission | 74.0 ± 5.1 | 75.0 ± 7.3 | 70.5 ± 5.7 | 77.3 ± 8.6 |
| Discharge | 72.3 ± 4.3 | 69.8 ± 5.5 | 69.8 ± 4.2 | 73.4 ± 8.0 |
| Supine | | | | |
| Systolic | 110.5 ± 8.5 | 108.8 ± 6.0 | 110.5 ± 11.4 | 107.8 ± 10.8 |
| Admission | | | | |
| Discharge | 105.5 ± 6.7 | 102.5 ± 11.2 | 106.8 ± 9.9 | 108.6 ± 10.8 |
| Diastolic | 69.3 ± 6.0 | 69.8 ± 3.1 | 67.8 ± 6.5 | 67.3 ± 6.9 |
| Admission | | | | |
| Discharge | 67.0 ± 4.5 | 65.3 ± 7.1 | 68.3 ± 5.2 | 68.9 ± 5.0 |
| Pulse | | | | |
| Admission | 70.3 ± 7.5 | 71.5 ± 6.7 | 72.8 ± 4.7 | 75.5 ± 10.9 |
| Discharge | 68.8 ± 4.7 | 66.8 ± 5.4 | 68.3 ± 5.9 | 72.6 ± 7.4 |

**Standard deviation

TABLE 13
VITAL SIGNS
Means of Within-Subject Differences Between Pre-Treatment
and Post-Treatment at Each Visit

| | PLACEBO | PGE₁ 0.05% | PGE₁ 0.1% | PGE₁ 0.2% |
|---|---|---|---|---|
| | (Visit 2) | (Visit 3) | (Visit 4) | (Visit 5) |
| | N = 8 | N = 8 | N = 8 | N = 7 |
| Standing | | | | |
| Systolic | 1.25 ± 3.0* | −4.00 ± 4.3 | 0.50 ± 4.7 | −4.57 ± 4.2 |
| | (p = 0.68) | (p = 0.39) | (p = 0.92) | (p = 0.32) |
| Diastolic | 1.50 ± 1.8 | −4.75 ± 3.5 | −0.75 ± 3.0 | −3.71 ± 2.3 |
| | (p = 0.42) | (p = 0.22) | (p = 0.81) | (p = 0.16) |
| Pulse | −1.75 ± 2.7 | −5.25 ± 2.7 | −0.75 ± 2.6 | −5.71 ± 2.7 |
| | (p = 0.54) | (p = 0.09) | (p = 0.78) | (p = 0.08) |
| Supine | | | | |
| Systolic | −5.00 ± 2.2 | −6.25 ± 2.9 | −3.75 ± 3.9 | −0.57 ± 4.0 |
| | (p = 0.055) | (p = 0.07) | (p = 0.37) | (p = 0.89) |
| Diastolic | −2.25 ± 1.2 | −4.50 ± 2.5 | 0.50 ± 1.9 | 0.57 ± 3.5 |
| | (p = 0.11) | (p = 0.11) | (p = 0.80) | (p = 0.88) |
| Pulse | −1.50 ± 3.7 | −4.75 ± 2.7 | −4.50 ± 2.2 | −4.86 ± 3.4 |
| | (p = 0.70) | (p = 0.12) | (p = 0.08) | (p = 0.20) |

*Standard error

The minimal adverse events and the comparison of vital signs (Tables 12 and 13) indicated that the medication was well tolerated.

The raw data are presented in Table 14, below. Baseline response levels were recorded following the administration of placebo (visit 2). Maximum response levels were recorded following the administration of PGE₁ at 0.05%, 0.1% or 0.2% (visits 3-5). For all questions, the maximum responses showed increased means compared to baseline means (Table 14, bottom row).

The results of analysis of the raw data of Table 14 are presented in Table 15, below, where statistical significance at the p<0.05 level is designated by “*” and at the p<0.01 level by “**”. All statistical analysis of the difference between the mean baseline value and the mean maximum response were performed by paired t test with two-tailed P value as described in Bancroft, H.; 1957, Introduction to Biostatistics, 172-182.

The increases in maximum response means compared to baseline means were statistically significant for Questions No. 2, No. 4, No. 5, and No. 6 as shown in Table 15. These results indicate that PGE₁ treatment significantly increased female sexual response including subjective arousal, engorgement, vaginal tingling, and pleasurable feelings. The significant (p<0.05) response to question No. 2, “How much subjective arousal did you have during this video?”, shows that the method of the invention is effective in increasing subjective arousal in even this small sample of treated patients. The significant (p<0.01) response to question No. 4, “How much engorgement (fullness) did you feel during this video?”, shows that the method of the invention is effective in subjective awareness of increased engorgement in even this small sample of treated patients. The significant (p<0.01) response to question No. 5, “How much vaginal tingling did you feel during this video?” shows that the method of the invention is effective in increasing subjective awareness of increase vaginal tingling in even this small sample of treated patients. The significant (p<0.05) response to question No. 6, “How pleasurable were the feelings you had during the presentation of this video?” shows that the method of the invention is effective in increasing subjective pleasurable feelings in even this small sample of treated patients. These data thus show significant increases in subjective aspects that are specific to female sexual arousal disorder.

TABLE 14
Questionnaire Response Raw Data

| | Question | | Question | | Question | | Question | | Question | | Question | |
|---|---|---|---|---|---|---|---|---|---|---|---|---|
| | #2 | | #3 | | #4 | | #5 | | #6 | | #9 | |
| Patient | Base- | Max. | Base- | Max. | Base- | Max. | Base- | Max. | Base- | Max. | Base- | Max. |
| No. | line | Resp. | Line | Resp. | line | Resp. | line | Resp. | line | Resp. | line | Resp. |
| 1 | 3 | 7 | 3 | 7 | 2 | 6 | 1 | 5 | 3 | 7 | 3 | 7 |
| 2 | 3 | 4 | 3 | 4 | 1 | 1 | 0 | 0 | 4 | 4 | 9 | 10 |
| 3 | 6 | 7 | 8 | 6 | 2 | 7 | 4 | 5 | 7 | 8 | 9 | 10 |
| 4 | 7 | 6 | 5 | 6 | 7 | 8 | 6 | 6 | 6 | 6 | 2 | 8 |
| 5 | 3 | 5 | 2 | 5 | 2 | 5 | 3 | 5 | 4 | 6 | 6 | 8 |
| 6 | 4 | 5 | 7 | 8 | 1 | 6 | 0 | 3 | 6 | 6 | 9 | 7 |
| 7 | 4 | 5 | 4 | 5 | 1 | 3 | 2 | 4 | 2 | 4 | 7 | 10 |
| 8 | 3 | 5 | 2 | 3 | 1 | 2 | 0 | 4 | 2 | 6 | 7 | 7 |
| Mean | 4.1 | 5.5 | 4.3 | 5.5 | 2.1 | 4.8 | 2.0 | 4.0 | 4.3 | 5.9 | 6.5 | 8.4 |

TABLE 15
Statistical Analysis of Responses (p value)

| Question #2 | Question #3 | Question #4 | Question #5 | Question #6 | Question #9 |
|---|---|---|---|---|---|
| P = 0.028 * | P = 0.0835 | P = 0.0062 ** | P = 0.0096 ** | P = 0.0284 * | P = 0.0693 |

Statistical significance at the p < 0.05 level is designated by “*” and at the p < 0.01 level by “**”.

### EXAMPLE 6

### A Randomized, Placebo-Controlled, Double-Blind, Parallel Design Study of the Efficacy and Safety of PGE₁ Cream in Pre-Menopausal Patients with FSAD

The purpose of this study was to evaluate the efficacy and safety of three doses of PGE₁ cream, compared to a placebo cream, under conditions of home use in conjunction with vaginal intercourse in pre-menopausal women with female sexual arousal disorder (FSAD).

Study Design

This was a multicenter, randomized, double-blind, placebo-controlled, parallel-group study. Following a screening visit to assess sexual problems, anxiety, depression, and safety measures, patients were randomly assigned to one of four treatment groups: placebo cream, 0.5 mg PGE₁ cream, 1.0 mg PGE₁ cream or 1.5 mg PGE₁ (alprostadil) cream. Patients were then given 10 double-blind doses of study medication for use at in the subsequent 6 weeks. Patients were given instructions to apply the medication directly to the vulva tissues and clitoris using their finger. Patients were also given diaries for recording information regarding sexual encounters and arousal levels. After approximately 6 weeks, the patients returned to the study site for final efficacy and safety evaluations.

Number of Patients (Planned and Analyzed)

Ninety-eight patients were screened, enrolled, and randomly assigned to a dose group. Four patients who took study medication home with them either were lost to follow-up or decided not to participate; therefore, 94 patients who were known to have taken at least one dose of study medication were included in the safety population. Each of these patients also provided efficacy data and was, therefore, included in the intent-to-treat (ITT) cohort for all efficacy analyses.

Diagnosis and Main Criteria for Inclusion

Women 21 to 50 years of age who were pre-menopausal, non-nursing, and not pregnant, who were using hormonal contraception or had undergone tubal ligation, and who met diagnostic criteria for FSAD for at least the preceding 6 months.

Description of Measurements

The FSFI is a validated 19-item questionnaire used to assess female sexual activity. (Derogatis L, for American Foundation of Urologic Disease, Inc.: Female Sexual Distress Scale, preliminary report. American Urological Association Annual Meeting, 2000). Individual items are rated on a 6-point scale from 0 to 5, or on a 5-point scale from 1 to 5, with higher scores correlated with a state of normal sexual health. Each question evaluates one of six domains: desire (questions 1 and 2), arousal (questions 3-6), lubrication (questions 7-10), orgasm (questions 11-13), satisfaction (questions 14-16), and pain (questions 17-19).

The FSDS is a 20-item sexual distress inventory. For each item, the patient circles a number from 0 (never) to 4 (always) that best describes how often that problem has bothered her or caused her distress during the preceding 6 weeks.

The GAQ, “While using the study medication, did you feel that your level of sexual arousal (excitement) improved?” is a type of global tool that is utilized widely in clinical trials of sexual dysfunction. Possible responses to the GAQ ranged from 1 (very much worse) to 7 (very much better).

The FSEP is a 6-item questionnaire completed after each sexual encounter. Four questions are answered yes or no: (Question #1) were the conditions right for a satisfactory sexual intercourse, (Question #3) were you satisfied with your arousal [excitement] during this sexual encounter, (Question #4) did you achieve enough lubrication to allow comfortable intercourse, and (Question #5) did you achieve orgasm? An affirmative answer is assigned a score of 1. Question #2 asks for details about the sexual activity (no genital stimulation, genital stimulation without intercourse, or genital stimulation with intercourse). Question #6 asks the subject to rate her level of arousal on a 4-point scale from “not at all aroused” to “completely aroused”.

Efficacy Endpoints

The primary efficacy endpoint was the success rate for patient satisfaction with sexual arousal during intercourse, as reported in the patient diaries. The success rate was defined as the number of “yes” responses to Question 3 of the FSEP questionnaire, “Were you satisfied with your arousal [excitement] during this sexual encounter?” divided by the number of attempts at sexual encounters.

The secondary efficacy endpoints were the change from baseline in scores for the Female Sexual Distress Scale (FSDS); the Female Sexual Function Index (FSFI); the Global Assessment Question (GAQ), and the scores for the remaining questions in the FSEP.

Safety was assessed by monitoring adverse events and the use of concomitant medications, as well as evaluating changes in clinical laboratory tests, vital signs, electrocardiograms (ECGs), and physical examinations.

Statistical Methods

The primary efficacy endpoint, changes from baseline in the FSDS domain and total scores, changes from baseline in the FSFI individual items and total scores, mean GAQ scores, and success rates for the remaining FSEP questions were analyzed using an analysis of variance (ANOVA) model with treatment as a factor. A chi-square test was used to analyze the distribution of GAQ responses into two categories: no improvement or change (GAQ score=1 to 4) and improvement (GAQ score=5 to 7). Demographic and baseline characteristics, study medication usage, and changes in vital signs after administration of the test dose were analyzed using either ANOVA models (continuous variables) or Fisher's exact test (categorical variables).

Patient Disposition

Of the 98 patients who were randomly assigned to a treatment group, 21 patients (91%) in the placebo group, 24 (89%) in the 500 mcg group, 24 (92%) in the 1000 mcg group, and 16 (73%) in the 1500 mcg group completed the study. The most common reason for early discontinuation was adverse events, which led to the withdrawal of 1, 1, 1, and 4 patients of each group, respectively. The ITT and safety populations included 23 patients who used placebo, 25 who used 0.5 mg PGE₁ cream, 25 who used 1.0 mg PGE₁ mcg, and 21 who used 1.5 mg PGE₁ cream.

Efficacy Results

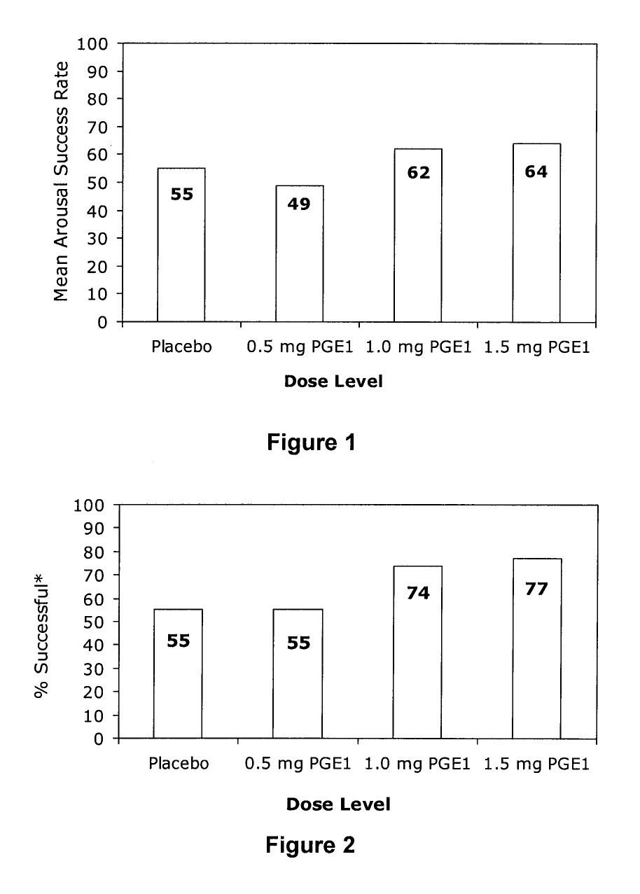

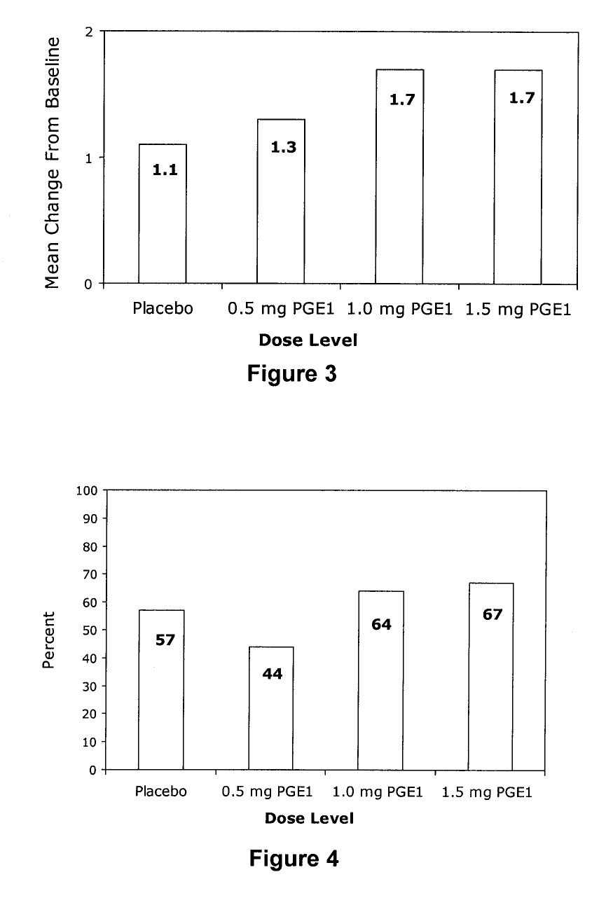

There was no statistically significant difference among the treatment groups in the results for the primary efficacy variable, and no difference between any active treatment group and placebo. The lack of statistical significance is primarily due to the fact that the placebo group in this study had an overall high response rate. A more careful selection of patients is needed and/or perhaps the placebo contributed to a positive response due to the inherent lubrication properties of the cream formulation and the method of application (to the entire external vaginal tissues including clitoris). However, an examination of the data in patients that had satisfactory participation in the study (at least 3 attempts at a sexual encounter) clearly shows a dose dependent trend (FIGS. 1, 2, 3 and 4). FIG. 1 is a graphical representation of the results of studies of Example 6 showing the primary efficacy measure, mean arousal success rate for the patients of the ITT population who made at least 3 sexual encounter attempts. FIG. 2 is a graphical representation of the results of studies of Example 6 showing the percent of women in each group who attempted intercourse at least 3 times out of the possible 10 times during the 6 week study and achieved satisfactory arousal during at least 50% of their attempts. FIG. 3 is a graphical representation of the results of studies of Example 6 showing the mean change from baseline of the FSFI Arousal Domain. FIG. 4 is a graphical representation of the results of studies of Example 6 showing efficacy results as measured by the Global Assessment Question (GAQ) “While on the study medication did you feel your sexual arousal improve?”

TABLE 16
Summary of Efficacy Results (ITT Population)

| | | 0.5 mg | 1.0 mg | 1.5 mg |
|---|---|---|---|---|
| | Placebo | PGE₁ | PGE₁ | PGE₁ |
| Number of patients | 23 | 25 | 25 | 21 |
| Arousal Success rate (%)¹ | 55 | 49 | 62 | 64 |
| [FSEP Question 3] | | | | |
| Mean change from baseline in | 1.1 | 1.3 | 1.7 | 1.7 |
| FSFI Arousal Domain Score | | | | |
| Mean change from baseline in | −17 | −15 | −20 | −16 |
| FSDS total score | | | | |
| Had improvement in level of | 57 | 44 | 64 | 67 |
| sexual arousal (%) [GAQ] | | | | |

¹In patients that made at least 3 sexual encounter attempts

Safety Results.

A summary of treatment related adverse events is presented in Table 17.

TABLE 17
Summary of treatment related adverse events

| | | 0.5 mg | 1.0 mg | 1.5 mg |
|---|---|---|---|---|
| | Placebo | PGE₁ | PGE₁ | PGE₁ |
| Number of Patients | 23 | 25 | 25 | 21 |
| Treated | | | | |
| Number (%) of patients | 2 (9%) | 12 (48%) | 9 (36%) | 12 (57%) |
| with at least one AE | | | | |
| Number (%) of patients | 0 | 1 (4%) | 0 | 4 (19%) |
| withdrawn due to AEs | | | | |
| Number of serious AEs | 0 | 0 | 0 | 0 |

^{a} An adverse event was considered treatment-related if the investigator categorized it as definitely, probably, or possibly related to study medication.

There were no apparent effects of any dose of PGE₁ cream on clinical laboratory tests, ECGs, or physical examinations. Patients in the 1.5 mg PGE₁ dose group showed nonsignificant decreases from baseline in systolic and diastolic blood pressure at the final evaluation; however, the treatment groups were not balanced at baseline for diastolic blood pressure. A nonsignificant change from baseline in pulse was observed in the placebo group.

Conclusions: Even though statistical significance was not achieved with the primary efficacy endpoints, the data indicate a dose dependent efficacy rate profile despite a high placebo effect (FIGS. 1, 2, 3 and 4). The safety results suggest that these doses were well tolerated, but as expected topical adverse effects were found to be dose dependent.

### EXAMPLE 7

### Further Clinical Study in Women Suffering from Female Sexual Arousal Disorder

A randomized, placebo-controlled double blind crossover clinical efficacy and safety study was performed on eleven female patients 21-45 years of age. The patients who were selected for the study had been diagnosed with Female Sexual Arousal Disorder (FSAD) of more than 6 months duration.

The ingredients of the study medications are summarized in Table 18, below.

Composition A was a topical prostaglandin E₁ composition comprising 0.2 weight-percent prostaglandin E₁. The placebo used, Composition B, was the composition lacking the active ingredient prostaglandin E₁. A dose of 500 mg was packaged in a single-use applicator.

TABLE 18
Composition of Study Medications

| Ingredient | (% w/w) |
|---|---|
| Ethyl Alcohol, dehydrated, USP | 5.0 |
| DDAIP HCl | 1.6 |
| Ethyl Laurate, FCC | 3.0 |
| Guar Gum, modified | 2.5 |
| Sodium Hydroxide Solution, 1M | Qs pH 5.5 |
| | (adjust pH of cream) |
| Sterile Water for Injection, USP, buffered | qs 100 |
| to pH 5.5 with 0.1M Potassium | |
| Phosphate Monobasic, NF | |

To 450 milligrams of the above cream base was added 50 milligrams of polyethylene glycol (PEG 400) that contained 1 mg prostaglandin E₁ to make Composition A. 50 milligrams of polyethylene glycol (PEG 400) alone was added to 450 milligrams of the above cream base to make Composition B (placebo), which contained no prostaglandin E₁.

The patients were instructed regarding administration of the topical composition. The recommended dosage was stated as no more than one applicator per day. The patients were instructed to apply the cream as directed by the physician approximately 5-20 minutes prior to attempting intercourse. The patients were told to micturate and wash their hands and vulva by using soapsuds before applying the topical composition. After removing the cap from the tip of the applicator, the patient was to expel the cream onto a fingertip; and equally spread the cream over the clitoris. The patients were directed to spread the remaining cream into vaginal anterior G-spot at about 5 cm deep, 1/3 of the middle passage of the vagina. Each patient was asked to apply one dose of Composition A and one dose of Composition B (placebo) during the course of the study. Either Composition A or Composition B (placebo) was applied first, with a waiting period of at least five days before the application of the second study medication. The patients were asked to keep a diary, and note whether the use of the medication produced an improvement. Contact was lost with three of the eleven patients. No adverse effects were reported in the remaining population of eight patients. No patient diaries were returned by three patients; of these, two patients reported that neither composition was effective, while the other patient reported that Composition A but not B (placebo) was effective. Overall, five of the eight patients reporting indicated that Composition A, containing 0.2 weight percent prostaglandin E₁, was effective. Only one of the eight patients reported that the placebo was effective; she also reported that composition A was effective. Three patients reported that neither composition was effective. No patients reported that composition B was effective, but not composition A. While the size of the study population is small, the difference between the reported efficacy of composition A (5/8 or 62.5%) and the reported efficacy of composition B (1/8 or 12.5%) is significantly different at the P≦0.01 level. Thus, in this limited preliminary study, the composition containing the active ingredient, prostaglandin E₁, was found to be more effective than the placebo when applied to the clitoris and intravaginally to the Grafenberg spot (G-spot) about 5-20 minutes prior to intercourse.

### EXAMPLE 8

### Large Double-Blind Clinical Study

A multi-center, randomized, double-blind, placebo-controlled, parallel design, non-treatment baseline Phase 3 study was performed to investigate the efficacy and safety of an embodiment of the present invention compared to placebo in pre-menopausal and postmenopausal women with Female Sexual Arousal Disorder (FSAD). Three hundred and sixty-three women completed the nine-month study performed at four study sites in China (Beijing University First Hospital, Beijing University Third Hospital, Beijing Xuanwu Hospital, and Beijing Chaoyang Hospital).

The objective of this study was to evaluate the safety and efficacy of 0.4% alprostadil cream in increasing dosages (placebo; 0.5 mg PGE₁, 0.7 mg PGE₁ and 0.9 mg PGE₁, corresponding to doses of 125 mg, 175 mg, and 225 mg of cream that also contained 0.5% weight percent of dodecyl N,N-dimethylamino isoproprionate (DDAIP HCl).

Study Objectives

To evaluate the clinical efficacy and safety of PGE₁ cream (alprostadil cream) for the treatment of female sexual arousal disorder (FSAD) in women.

Study Design and Duration

This is a multicenter, randomized, double-blind, placebo-controlled, parallel design study to examine the efficacy and safety of alprostadil cream versus placebo in women with FSAD. Patients were randomly assigned to receive placebo, 0.5 mg, 0.7 mg or 0.9 mg alprostadil (PGE₁) during 2 evaluation periods of active treatment, following a 4-week non-treatment baseline period. The study was expected to be completed in approximately 4-month treatment period on average. The period between visits was about thirty days for the duration of the treatment periods. Each patient was expected to attempt sexual intercourse about 3-5 times during a treatment period.

Dosage, Route of Administration, Dosing Regimen

The PGE₁ cream formulation containing either, placebo, 0.5 mg, 0.7 mg or 0.9 mg PGE₁ alprostadil, administered to the clitoris and G-spot. Ten (10) doses per patient were administered at home over the 2 evaluation periods (i.e., 5 doses per patient for each treatment period). The patient was instructed to engage in foreplay with her partner to facilitate the action of the medication. Patients who received study medication for at-home use were instructed to attempt vaginal sexual intercourse after application of the test article on ten separate occasions (no more than once a day) during treatment intervals.

Primary Safety Measurements

Safety was assessed by monitoring adverse events and changes in vital signs, clinical laboratory test results, ECG and physical examinations.

Primary Efficacy Measurement

The primary efficacy end point is the change in arousal success rate (baseline versus treatment period), defined as the number of successes (patient satisfaction with sexual arousal during sexual activity as indicated by a “Yes” response to diary question #3 of the female sexual encounter profile [FSEP]) divided by the number of sexual encounters.

Secondary Efficacy Measurement

The secondary efficacy end points are the responses to a global assessment question (GAQ), the responses to the remaining diary questions (FSEP), and changes in the responses (baseline versus final visit) to the Female Sexual Distress Scale (FSDS) and to the Female Sexual Function Index (FSFI) among groups.

Data Analysis

The primary efficacy end point is the change in arousal success rate (baseline versus treatment period), defined as the number of successes (patient satisfaction with sexual arousal during sexual activity as indicated by a “Yes” response to diary question #3 [FSEP]) divided by the number of sexual encounters. Comparisons for the primary efficacy end point were made among treatment groups. The secondary efficacy end points are the responses to a global assessment question (GAQ), the responses to the remaining diary questions (FSEP), and changes in the responses (baseline versus final visit) to the Female Sexual Distress Scale (FSDS) and Female Sexual Function Index (FSFI). Comparisons were made among treatment groups for all secondary efficacy end points. The arousal success rate of each visit was calculated and statistically described. The rank-sum test was used to determine the difference between groups.

Primary Efficacy Analysis of the Intent-To-Treat (ITT) population shows a significant increase in arousal success rates with dose. Secondary Efficacy Analysis of the ITT population at the end of the second evaluation period shows a significant increase in FSFI scores versus placebo at the 0.9 mg doses. A significant decrease in distress while on the drug is reflected in the data, which shows significant differences from placebo at the 0.9 mg doses. This is an important finding since stress is an integral component of female sexual arousal disorder. The analysis of the Global Assessment Question scores—a measurement of the patient perceived improvement in arousal—shows a significant improvement versus placebo at all dose levels for the PGE₁ cream. The data generated in this study shows dose related improvement in all endpoints over the whole course of therapy.

The primary efficacy variable was the arousal success rate. The arousal success rate of each visit was calculated and statistically described. The Wilcoxon test was used to determine the differences between groups and the total arousal success rate of the two treatment periods was also calculated. The second treatment period was the principal evaluation period. The arousal success rate was calculated and classified by 25%, 50%, and 75%, and the N/Percent of each class was described. The primary measure of efficacy for this study was the change in arousal success rate (Non-treatment period [baseline] versus treatment period) as defined by the number of successes (patient satisfaction with sexual arousal during sexual activity as indicated by a “Yes” response to diary question #3 of the Female Sexual Encounter Profile [FSEP]) divided by the number of sexual encounters. The secondary measures of efficacy for this study were change in scores (baseline versus final visit) for the Female Sexual Distress Scale (FSDS), scores reported in the Global Assessment Questionnaire (GAQ), scores reported in the FSEP, and changes in responses (baseline versus final visit) to the Female Sexual Function Index (FSFI) among groups. The study involved four clinic visits. A total of 400 patients were enrolled and randomly assigned to the four treatment groups. Patients whose arousal success rates were over 80% in the screening period were excluded from the ITT and PP populations. Table 19 summarizes the disposition of patients during the course of the study.

TABLE 19
Patient Disposition

| | | 0.5 mg | 0.7 mg | 0.9 mg | |
|---|---|---|---|---|---|
| | Placebo | PGE₁ | PGE₁ | PGE₁ | Total |
| Screened | — | — | — | — | 437 |
| Randomized | 100 | 100 | 100 | 100 | 400 |
| Completed | 95 | 92 | 92 | 93 | 372 |
| Discontinued | 5 | 8 | 8 | 7 | 28 |
| Reasons for | | | | | |
| discontinuation | | | | | |
| Adverse events | | | 2 | 3 | 5 |
| Protocol deviation | 0 | 0 | 0 | 0 | 0 |
| Patient withdrew consent | 1 | 0 | 1 | 0 | 2 |
| Laboratory abnormality | | | | 1 | 1 |
| Lost to follow-up | 3 | 5 | 1 | 2 | 11 |
| Others | 1 | 3 | 4 | 1 | 9 |
| Safety Population^{a} | 98 | 94 | 97 | 98 | 387 |
| Intent-to-treat | 97 | 91 | 91 | 95 | 374 |
| (ITT)^{b} population | | | | | |
| Per-Protocol (PP) | 94 | 89 | 88 | 92 | 363 |
| population | | | | | |

^{a}All patients who used at least one dose of study medication
^{b}All patients who used at least three doses of study medication and had at least one baseline and valid post-baseline efficacy evaluation

Demographic and baseline characteristics for the ITT population were summarized. Continuous variables were summarized using descriptive statistics (N, mean, median, standard deviation [SD], minimum, and maximum), and categorical variables were summarized by frequency distribution and percent. Demographics and baseline characteristics included age, gender, race, height, weight, allergic history, drinking, smoking, duration of FSAD, medical history, previous medication, and concomitant medications. Previous and concomitant medications were coded to a therapeutic area according to the variables character; the ANOVA/Kruskall-Wallis test was used to determine the difference of age, height, weight and other baseline quantificational characteristics between different groups. The X2 (chi-square) test was used to determine the differences of ethnic background, allergy history, alcohol use, smoking, duration of FSAD, medical history, previous medication, concomitant medications and other categorical variables between different groups.

A summary of patient demographics is presented in Table 20. Out of 374 patients in the ITT population, most patients (94.4%) were of Han ethnic background. The overall mean age was 45.0 years, and the mean weight and height were 59.7 kg and 161.1 cm, respectively. A total of 67 (16.8%) patients reported allergy history, 17 (4.2%) patients reported alcohol use, and 4 (1.0%) patients reported smoking. There were no statistically significant differences between the groups in these baseline characteristics.

TABLE 20
Demographics (ITT Patients)

| | Placebo | 0.5 mg PGE₁ | 0.7 mg PGE₁ | 0.9 mg PGE₁ |
|---|---|---|---|---|
| Number of | 97 | 91 | 91 | 95 |
| patients | | | | |
| Age (years) | | | | |
| Mean | 45.2 | 45.8 | 44.6 | 44.4 |
| SD | 7.31 | 8.76 | 8.27 | 8.36 |
| Range | 22.3-61.9 | 22.3-61.9 | 26.4-60.4 | 23.6-62.1 |
| Height (cm) | | | | |
| Mean | 161.4 | 160.7 | 161.0 | 161.2 |
| SD | 4.72 | 5.62 | 4.78 | 5.03 |
| Range | 150-173 | 148-170 | 140-175 | 150-173 |
| Weight (kg) | | | | |
| Mean | 60.6 | 59.0 | 59.8 | 59.4 |
| SD | 8.9 | 7.8 | 8.4 | 8.0 |
| Range | 40-86 | 39-86 | 40-85 | 45-92 |
| Race, (%) | | | | |
| Han | 89 (97.8%) | 89 (97.8%) | 86 (94.5%) | 85 (89.5%) |
| Others | 4 (4.1%) | 2 (2.2%) | 5 (5.5%) | 10 (10.5%) |

Baseline scores of the Female Sexual Distress Scale (FSDS, day 0) and Female Sexual Function Index (FSFI, day 0) are shown in Table 21. There was no significant difference in FSDS or FSFI scores between groups.

TABLE 21
Baseline Assessments (ITT Patients)

| | | | | | p-Value |
|---|---|---|---|---|---|
| | Placebo | 0.5 mg PGE₁ | 0.7 mg PGE₁ | 0.9 mg PGE | (F test) |
| Number of patients | 97 | 91 | 91 | 95 | |
| FSDS Total Score | 50.84 ± 9.65 | 50.51 ± 8.72 | 51.88 ± 9.82 | 51.87 ± 9.70 | 0.6703 |
| FSFI Total Score | 36.64 ± 11.16 | 38.66 ± 10.51 | 39.18 ± 11.50 | 36.80 ± 10.20 | 0.2679 |
| FSFI Desire Factor | 3.96(1.48) | 4.04(1.63) | 3.74(1.49) | 3.91(1.47) | 0.5732 |
| FSFI Arousal Factor | 7.47(2.59) | 6.96(2.24) | 6.73(2.53) | 7.02(2.40) | 0.1997 |
| FSFI Lubrication | 8.68(3.70) | 8.15(3.21) | 8.31(3.10) | 8.98(3.60) | 0.3480 |
| Factor | | | | | |
| FSFI Orgasm Factor | 5.58(2.40) | 5.12(2.09) | 4.92(1.81) | 5.02(2.04) | 0.1443 |
| FSFI Satisfaction | 6.87(2.36) | 6.59(2.17) | 6.60(2.30) | 6.88(1.90) | 0.6842 |
| Factor | | | | | |
| FSFI Pain Factor | 6.62(2.95) | 5.93(2.76) | 6.34(2.91) | 6.85(3.00) | 0.1646 |

The arousal success rate of each visit was calculated and statistically described. The Rank-Sum test was used to determine the difference between groups. The total arousal success rate of the two treatment periods was also calculated. The second treatment period was the principal evaluation period. The analysis of arousal success rate for the ITT population is presented in Table 22.

Results for ITT population: Arousal success rate for the first treatment period vs. baseline was 16.99, 32.00, 27.51 and 38.65% for the placebo, 0.5 mg PGE₁, 0.7 mg PGE₁, and 0.9 mg PGE₁ groups, respectively. The arousal success rate increased during the second treatment period vs. baseline: and was 28.58, 41.94, 40.28 and, 51.41% for the placebo, 0.5 mg PGE₁, 0.7 mg PGE₁, and 0.9 mg PGE₁ groups, respectively. The arousal success rate increased during the entire treatment period vs. baseline and was: 22.63, 36.67, 34.01 and 44.29 for the placebo, 0.5 mg PGE₁, 0.7 mg PGE₁, and 0.9 mg PGE₁ groups, respectively. The arousal success rate for every treatment period for all four groups was significantly increased vs. baseline. Results for the PP population were similar to those for the ITT population. Aside from study center 4, the arousal success rate for all alprostadil treatment groups were higher than placebo. However for study center 4, the arousal success rate increase for the placebo group was higher than those of the 0.5 mg PGE₁, and 0.7 mg PGE₁, groups but lower than that of the 0.9 mg PGE₁ group.

Few of the patients achieved an arousal success rate over 75% or 50%. In the first treatment period, the arousal success rates of patients whose arousal rates were over 75% were 18.56, 26.37, 25.27, and 37.89% in the placebo, 0.5 mg PGE₁, 0.7 mg PGE₁, and 0.9 mg PGE₁ groups, respectively. The patients with an arousal success rate≧75% were 8.25, 19.78, 18.68, and 28.42% vs. baseline in the placebo, 0.5 mg PGE₁, 0.7 mg PGE₁, and 0.9 mg PGE₁ groups, respectively. In the second treatment period, the arousal success rates of patients whose arousal rates were over 75% were 27.96, 41.11, 41.57, and 52.17% in the placebo, 0.5 mg PGE₁, 0.7 mg PGE₁, and 0.9 mg PGE₁ groups, respectively. The patients with an arousal success rate increasing≧75% were 17.20, 34.44, 32.58 and, 39.13% vs. baseline in the placebo, 0.5 mg PGE₁, 0.7 mg PGE₁, and 0.9 mg PGE₁ groups, respectively. In the entire treatment period, arousal success rate in ≧75% of patients were 9.28, 19.78, 17.58, and 26.32% in the placebo, 0.5 mg PGE₁, 0.7 mg PGE₁, and 0.9 mg PGE₁ groups, respectively. The result of the arousal success rate of ≧50% of patients was similar to the result of the arousal success rate of ≧75% of the patients. The arousal success rate of the PP population were similar to those of the ITT population.

In the second treatment period, the P-values of the three hypotheses (i.e. the arousal success rate was the same between the 0.9 mg PGE₁ group and placebo group, the 0.7 mg PGE₁ group and placebo group, and the 0.5 mg PGE₁ group and the placebo group) were not all less than 0.05 two-sided. The P-value between the 0.9 mg PGE₁ group and placebo group was 0.0002 (two-sided); the corresponding null hypothesis was not met. The difference between the 0.9 mg PGE₁ group and placebo group was statistically significant (P-value 0.0002). Results for the PP population were similar to those of the ITT population.

For the entire treatment period, the P-values of the three hypotheses (i.e. the arousal success rate was the same between the 0.9 mg PGE₁ and placebo groups, the 0.7 mg PGE₁ and placebo groups, and the 0.5 mg PGE₁ and placebo groups) were all less than 0.05 (two-sided). The difference between all three alprostadil groups (0.5 mg PGE₁, 0.7 mg PGE₁, and 0.9 mg PGE₁) and the placebo group was statistically significant (P-value 0.0021). Results for the PP population were similar to those of ITT population.

TABLE 22
Arousal Success Rate (ITT Patients)

| | Placebo | 0.5 mg PGE₁ | 0.7 mg PGE₁ | 0.9 mg PGE₁ |
|---|---|---|---|---|
| Screening period (non-treatment period) | | | | |
| N (Missing) | 97 (0) | 91 (0) | 91 (0) | 95 (0) |
| Mean (SD) | 10.50(20.41) | 9.65 (17.51) | 9.45 (18.73) | 9.63 (16.91) |
| Min, Max | 0.00, 75.00 | 0.00, 66.67 | 0.00, 75.00 | 0.00, 75.00 |
| Median | 0.00 | 0.00 | 0.00 | 0.00 |
| p-Value (among-group, Rank Sum Test) = 0.9521 | | | | |
| First treatment period | | | | |
| N (Missing) | 97 (0) | 91 (0) | 91 (0) | 95 (0) |
| Mean (SD) | 27.49(37.32) | 41.65 (37.68) | 36.96 (38.85) | 48.28 (41.64) |
| Min, Max | 0.00, 100.00 | 0.00, 100.00 | 0.00, 100.00 | 0.00, 100.00 |
| Median | 0.00 | 40.0 | 25.00 | 50.00 |
| p-Value (cf placebo, ANOVA) | | 0.0068 | 0.0635 | 0.0004 |
| First treatment period - Screening period | | | | |
| N (Missing) | 97 (0) | 91 (0) | 91 (0) | 95 (0) |
| Mean (SD) | 16.99 (32.24) | 32.00 (39.14) | 27.51 (40.89) | 38.65 (43.74) |
| Min, Max | −66.67, 100.00 | −50.00, 100.00 | −66.67, 100.00 | −60.00, 100.00 |
| Median | 0.00 | 25.00 | 20.00 | 40.00 |
| p-Value (cf placebo, ANOVA) | | 0.0113 | 0.0855 | 0.0006 |
| Second treatment period | | | | |
| N (Missing) | 93 (4) | 90 (1) | 89 (2) | 92 (3) |
| Mean (SD) | 37.72 (40.37) | 51.15 (42.89) | 49.72 (43.41) | 60.92 (41.65) |
| Min, Max | 0.00, 100.00 | 0.00, 100.00 | 0.00, 100.00 | 0.00, 100.00 |
| Median | 25.00 | 60.00 | 60.00 | 75.00 |
| p-Value (cf placebo, ANOVA) | | 0.0410 | 0.0699 | 0.0003 |
| Second treatment period - Screening period | | | | |
| N (Missing) | 93 (4) | 90 (1) | 89 (2) | 92 (3) |
| Mean (SD) | 28.58 (39.39) | 41.94 (44.30) | 40.28 (46.11) | 51.41 (42.65) |
| Min, Max | −66.67, 100.00 | −35.00, 100.00 | −66.67, 100.00 | −60.00, 100.00 |
| Median | 20.00 | 41.67 | 40.00 | 57.50 |
| p-Value (cf placebo, ANOVA) | | 0.0533 | 0.0735 | 0.0002 |
| Entire treatment period | | | | |
| N (Missing) | 97 (0) | 91 (0) | 91 (0) | 95 (0) |
| Mean (SD) | 33.12 (36.02) | 46.32 (36.76) | 43.46 (36.49) | 53.92 (37.97) |
| Min, Max | 0.00, 100.00 | 0.00, 100.00 | 0.00, 100.00 | 0.00, 100.00 |
| Median | 25.00 | 50.00 | 42.86 | 57.14 |
| p-Value (cf placebo, ANOVA) | | 0.0161 | 0.0400 | 0.0002 |
| Entire treatment period - Screening period | | | | |
| N (Missing) | 97 (0) | 91 (0) | 91 (0) | 95 (0) |
| Mean (SD) | 22.63 (32.70) | 36.67 (38.44) | 34.01 (39.21) | 44.29 (39.66) |
| Min, Max | −66.67, 100.00 | 40.00, 100.00 | −66.67, 100.00 | −60.00, 100.00 |
| Median | 16.67 | 33.33 | 33.33 | 50.00 |
| p-Value (cf placebo, ANOVA) | | 0.0224 | 0.0358 | 0.0002 |

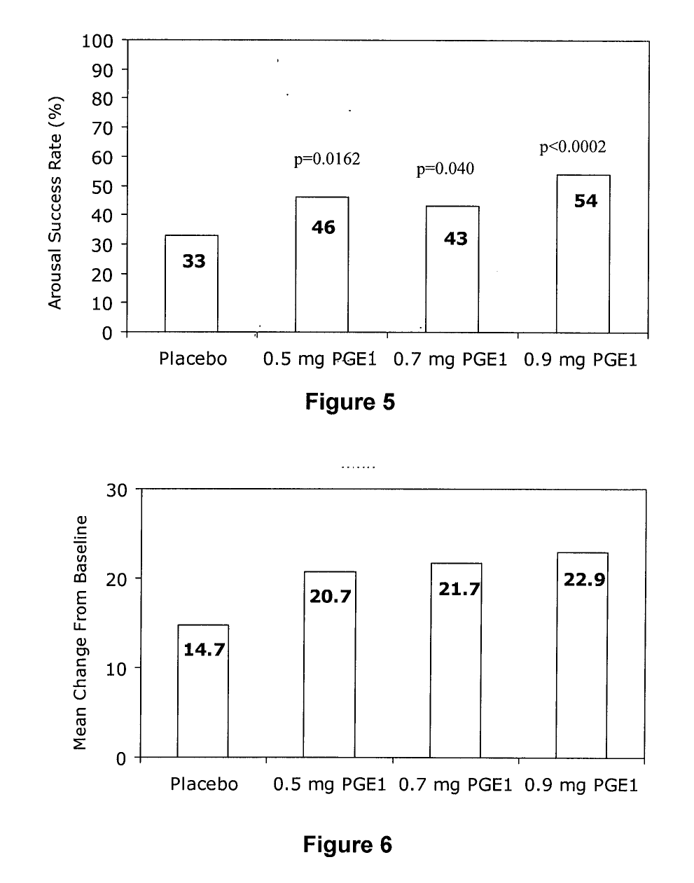

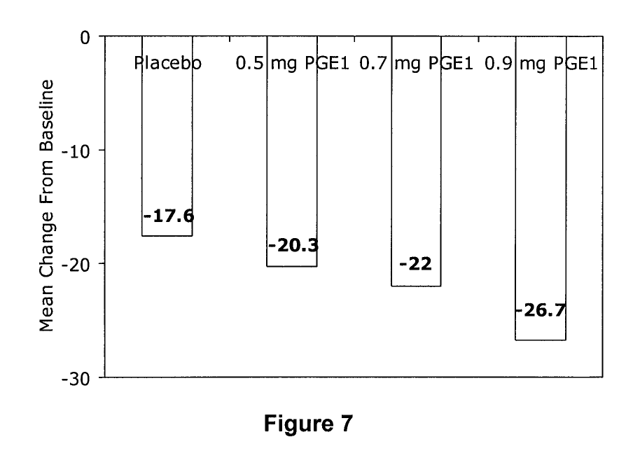

FIG. 5 is a graphical representation of the results of studies of Example 8 showing results from Question 3 of the FSEP, arousal success rate (Mean Percent of Attempts Resulting in Successful Arousal) for the four treatment groups. FIG. 6 is a graphical representation of the results of studies showing of Example 8 showing the mean change from baseline of the FSFI total score for the four treatment groups. FIG. 7 is a graphical representation of the results of studies of Example 8 showing the mean change from baseline of the FSDS score for the four treatment groups.

Subgroup Analysis: The population was grouped into two sub-groups, a postmenopausal group and a pre-menopausal group by E2 and FSH levels (E2<20 pg/ml and FSH≧40 pg/ml).

The postmenopausal group data for the ITT population is presented in Table 23. The arousal success rate increased during the first treatment period vs. baseline and was: 15.95, 31.72, 21.30 and 21.99% for the placebo, 0.5 mg PGE₁, 0.7 mg PGE₁, and 0.9 mg PGE₁ groups, respectively. The arousal success rate increase during the second treatment period vs. baseline was: 37.44, 42.53, 35.23, and 36.67% for the placebo, 0.5 mg PGE₁, 0.7 mg PGE₁, and 0.9 mg PGE₁ groups, respectively. The arousal success rate increase over the entire treatment period vs. baseline was: 24.84, 36.95, 26.99, and 29.14% for the placebo, 0.5 mg PGE₁, 0.7 mg PGE₁, and 0.9 mg PGE₁ groups, respectively. The difference between the entire treatment period vs. baseline for all four groups was not statistically significant. Results of PP population were similar to those of ITT population.

Arousal success rates were classified and described for the ITT population and the PP population. Few of the patients in the ITT population had an arousal success rate over 75%. In the first treatment period, the patients whose arousal success rates were over 75% were 25.00, 27.59, 21.74 and 19.23% for the placebo, 0.5 mg PGE₁, 0.7 mg PGE₁, and 0.9 mg PGE₁ groups, respectively. In the second treatment period, the rates of patients whose arousal rates were over 75% were 46.15, 44.83, 27.27 and 32.00% for placebo, 0.5 mg PGE₁, 0.7 mg PGE₁, and 0.9 mg PGE₁ groups, respectively. Over the entire treatment period the arousal success rates were 25.00, 34.48, 17.39, and 19.23% for the placebo, 0.5 mg PGE₁, 0.7 mg PGE₁, and 0.9 mg PGE₁ groups, respectively. Results for the PP population were similar to those of the ITT population.

TABLE 23
Arousal Success Rate, Postmenopausal ITT Patients

| | Placebo | 0.5 mg PGE₁ | 0.7 mg PGE₁ | 0.9 mg PGE₁ |
|---|---|---|---|---|
| N | 28 | 29 | 23 | 26 |
| Screening period, Mean (SD) | 14.40 | 8.33(16.62) | 5.22(12.38) | 9.62(15.20) |
| p-Value (among-group, Rank | | | | |
| Sum Test) = 0.5405 | | | | |
| First treatment period | 30.36(42.38) | 40.06(41.25) | 26.52(35.72) | 31.60(35.91) |
| p-Value (cf placebo, ANOVA) | | 0.3483 | 0.9665 | 0.6233 |
| First treatment period - | 15.95(29.64) | 31.72(42.64) | 21.30(36.31) | 21.99(34.72) |
| Screening period | | | | |
| p-Value (cf placebo, ANOVA) | | 0.1439 | 0.7372 | 0.4460 |
| Second treatment period | 48.78(46.14) | 50.86(46.57) | 39.77(39.99) | 45.07(42.95) |
| p-Value (cf placebo, ANOVA) | | 0.8927 | 0.4938 | 0.7505 |
| Second treatment period - | 37.44(43.00) | 42.53(48.20) | 35.23(41.03) | 36.67(40.78) |
| Screening period | | | | |
| p-Value (cf placebo, ANOVA) | | 0.7594 | 0.9914 | 0.9687 |
| Entire treatment period | 39.25(39.54) | 45.28(41.56) | 32.20(35.35) | 38.75(34.31) |
| p-Value (cf placebo, ANOVA) | | 0.6102 | 0.5923 | 0.9433 |
| Entire treatment period - | 24.84(31.01) | 36.95(43.18) | 26.99(36.68) | 29.14(32.68) |
| Screening period | | | | |
| p-Value (cf placebo, ANOVA) | | 0.3193 | 0.8932 | 0.6414 |

Results for the premenopausal group of the ITT population are presented in Table 24. The arousal success rate increase for the first treatment period vs. baseline was: 17.42, 32.12, 29.61, and 44.93% for the placebo, 0.5 mg PGE₁, 0.7 mg PGE₁, and 0.9 mg PGE₁ groups, respectively. The arousal success rate increase for the second treatment period vs. baseline was: 25.15, 41.67, 41.94, and 56.92% for the placebo, 0.5 mg PGE₁, 0.7 mg PGE₁, and 0.9 mg PGE₁ groups, respectively. The arousal rate increase for the entire treatment period vs. baseline was: 21.73, 36.54, 36.39, and 50.00% for the placebo, 0.5 mg PGE₁, 0.7 mg PGE₁, and 0.9 mg PGE₁ groups, respectively. The differences between the entire treatment period vs. baseline for all four groups were statistically significant. Results for the PP population were similar to those for the ITT population. The arousal success rates were classified and described (ITT population and PP population).

ITT population: Few patients achieved an arousal success rate over 75%. In the first treatment period, the percent of patients whose arousal success rate was ≧75% were 15.94, 25.81, 26.47 and 44.93% for the placebo, 0.5 mg PGE₁, 0.7 mg PGE₁, and 0.9 mg PGE₁ groups, respectively. During the second treatment period, the percent of patients whose arousal success rate was ≧75% were 20.90, 39.34, 46.27 and 59.70% for the placebo, 0.5 mg PGE₁, 0.7 mg PGE₁, and 0.9 mg PGE₁ groups, respectively. In the entire treatment period, the percent of patients whose arousal success rate was ≧75% were 18.84, 20.97, 27.94 and 46.38% for the placebo, 0.5 mg PGE₁, 0.7 mg PGE₁, and 0.9 mg PGE₁ groups, respectively. Results for the PP population were similar to those of the ITT population.

TABLE 24
Arousal Success Rate, Pre-menopausal ITT Patients

| | Placebo | 0.5 mg PGE₁ | 0.7 mg PGE₁ | 0.9 mg PGE₁ |
|---|---|---|---|---|
| N | 69 | 62 | 68 | 69 |
| Screening period, Mean (SD) | 8.91(18.45) | 10.27(18.01) | 10.88(20.32) | 9.64(17.61) |
| p-Value (among-group, Rank | | | | |
| Sum Test) = 0.8610 | | | | |
| First treatment period | 26.33(35.34) | 42.39(36.22) | 40.49(39.47) | 54.57(42.14) |
| p-Value (cf placebo, ANOVA) | | 0.0078 | 0.0290 | 0.0001 |
| First treatment period - | 17.42(33.43) | 32.12(37.76) | 29.61(42.38) | 44.93(45.34) |
| Screening period | | | | |
| p-Value (cf placebo, ANOVA) | | 0.0376 | 0.0784 | 0.0005 |
| Second treatment period | 33.43(37.40) | 51.28(41.43) | 52.99(44.27) | 66.84(39.88) |
| p-Value (cf placebo, ANOVA) | | 0.0160 | 0.0122 | 0.0000 |
| Second treatment period - | 25.15(37.68) | 41.67(42.74) | 41.94(47.84) | 56.92(42.31) |
| Screening period | | | | |
| p-Value (cf placebo, ANOVA) | | 0.0352 | 0.0372 | 0.0000 |
| Entire treatment period | 30.64(34.49) | 46.81(34.64) | 47.27(36.34) | 59.63(37.93) |
| p-Value (cf placebo, ANOVA) | | 0.0096 | 0.0062 | 0.0000 |
| Entire treatment period - | 21.73(33.53) | 36.54(36.39) | 36.39(40.01) | 50.00(40.75) |
| Screening period | | | | |
| p-Value (cf placebo, ANOVA) | | 0.0359 | 0.0185 | 0.0000 |

Analysis of the Secondary Efficacy Variable. For the ITT population, the FSFI scores for the first treatment period vs. baseline were 10.00, 14.54, 15.70, and 18.23 for the placebo, 0.5 mg PGE₁, 0.7 mg PGE₁, and 0.9 mg PGE₁ groups, respectively. In the second treatment period the change in FSFI score were 14.68, 20.71, 21.69, and 22.89 for the placebo, 0.5 mg PGE₁, 0.7 mg PGE₁, and 0.9 mg PGE₁ groups, respectively (see Table 23). Analysis of covariance of the results of the change in the FSFI score illustrated that the differences of score changes vs. baseline between the four groups was significant if the center and baseline effect was removed. If the interaction of drug and center was considered, the analysis of covariance result showed that there was an interaction effect between center and group (P<0.10).

TABLE 25
Female Sexual Function Index (FSFI) (Mean ± SD)
ITT Patients

| | Placebo | 0.5 mg PGE₁ | 0.7 mg PGE₁ | 0.9 mg PGE₁ |
|---|---|---|---|---|
| N | 97 | 91 | 91 | 95 |
| Visit 1 | 37.33(11.46) | 32.89(11.66) | 34.82(12.50) | 37.99(13.14) |
| (Screening) | | | | |
| Visit 2 | 39.18(11.50) | 36.80(10.20) | 36.64(11.16) | 38.66(10.51) |
| (Baseline) | | | | |
| Visit 3 | 49.18(17.78) | 51.34(17.28) | 52.34(16.68) | 56.89(18.92) |
| Visit 3 | 10.00(17.58) | 14.54(17.71) | 15.70(16.93) | 18.23(18.74) |
| (Baseline) | | | | |
| Visit 4 | 53.86(18.79) | 57.52(18.26) | 58.33(16.55) | 61.56(19.23) |
| Visit 4 | 14.68(20.36) | 20.71(17.91) | 21.69(17.95) | 22.89(19.77) |
| (Baseline) | | | | |
| p-Value | | 0.067 | 0.035 | 0.002 |

p-Value = Difference relative to placebo, from Analysis of Covariance method (the center and baseline effect was removed)

The increase in FSFI score for alprostadil treatment groups was higher than those for the placebo group in all study centers except study center 4. The differences between PGE₁ group treatment groups and the placebo group were analyzed by the same analysis of covariance (ANCOVA) method. The results are presented for the 0.9 mg PGE₁ group vs. placebo: The differences of FSFI score changes vs. baseline between the 0.9 mg PGE₁ group and placebo were significant if the study center and baseline effects were removed (P<0.05). The least-squares mean and 95% CI range of the FSFI score was: placebo group 15.93 (12.35-19.52); 0.9 mg PGE₁ group 23.83 (20.18-27.47); mean difference placebo was not significant if the center and baseline effect was removed. The least-squares mean and 95% CI range of the FSFI score was placebo group 16.30 (12.77-19.83), 0.5 mg PGE₁ group 20.94 (17.32-24.57); Mean difference between the placebo group vs 0.5 mg PGE₁ group was 4.64 (−0.34-9.62). The difference between the 0.7 mg PGE₁ group and the placebo group was significant if the center and baseline effect was removed. The least-squares mean and 95% CI range of the FSFI score was 0.7 mg PGE₁ group 21.96 (18.42-25.49); placebo group 16.78 (13.37-20.18); Mean difference between the 0.7 mg PGE₁ group vs placebo group was 5.18 (0.38-9.98). Thus, change in the FSFI scores of the 0.7 mg PGE₁ group was larger than that of the placebo group. Results of the PP population were similar to those of the ITT population. All aspects of the FSFI are presented in Table 24. Analysis demonstrated that differences between the 0.9 mg PGE₁ group and the placebo group were significant for each FSFI score.

TABLE 26
Female Sexual Function Index (FSFI) Score Changes
Visit 4 (Baseline) ITT Patients

| | Placebo | 0.5 mg PGE₁ | 0.7 mg PGE₁ | 0.9 mg PGE₁ |
|---|---|---|---|---|
| N | 97 | 91 | 91 | 95 |
| Desire | 1.16(2.26) | 1.44(2.04) | 1.84(2.27) | 2.00(2.08) |
| Factor | | | | |
| p-Value | | 0.185 | 0.104 | 0.002 |
| Arousal | 2.40(4.20) | 4.02(4.01) | 4.36(4.12) | 4.92(4.38) |
| Factor | | | | |
| p-Value | | 0.012 | 0.010 | 0.001 |
| Lubrication | 3.98(5.33) | 5.01(4.81) | 5.06(4.34) | 5.46(4.93) |
| Factor | | | | |
| p-Value | | 0.271 | 0.236 | 0.009 |
| Orgasm | 1.83(3.52) | 3.13(3.32) | 3.18(3.06) | 3.89(3.59) |
| Factor | | | | |
| p-Value | | 0.021 | 0.030 | 0.000 |
| Satisfaction | 1.80(3.81) | 3.13(3.09) | 3.43(2.97) | 3.27(3.08) |
| Factor | | | | |
| p-Value | | 0.014 | 0.002 | 0.001 |
| Pain Factor | 3.11(4.42) | 4.11(3.70) | 4.17(4.12) | 4.04(4.39) |
| p-Value | | 0.242 | 0.161 | 0.041 |

p-Value = Difference relative to placebo, from Analysis of Covariance method (the center and baseline effect was removed)

Female Sexual Distress Scale (FSDS) Results of the Analysis of Covariance illustrated that changes in the FSDS score between the four groups were significant if the study center and baseline effects were removed. The result of two-sided group analysis illustrated that the difference between the placebo and the 0.5 mg and 0.7 mg PGE₁ groups were not significant. The difference between the 0.9 mg PGE₁ group and the placebo group was significant. Details are listed in Table 27.

TABLE 27
Female Sexual Distress Scale (FSDS) (Mean ± SD) ITT Patients

| | Placebo | 0.5 mg PGE₁ | 0.7 mg PGE₁ | 0.9 mg PGE₁ |
|---|---|---|---|---|
| N | 97 | 91 | 91 | 95 |
| Visit 1 (Screening) | 51.74(7.90) | 52.88(9.19) | 52.57(9.00) | 52.22(9.05) |
| Visit 2 (Baseline) | 50.84(9.65) | 50.51(8.72) | 51.88(9.82) | 51.87(9.70) |
| Visit 3 | 40.53(17.93) | 35.84(17.61) | 38.09(18.61) | 33.45(18.52) |
| Visit 3 (Baseline) | −10.31(16.10) | −14.67(16.64) | −13.79(17.76) | −18.42(17.08) |
| Visit 4 | 33.24(20.32) | 30.23(18.74) | 29.90(19.34) | 25.91(18.79) |
| Visit 4 (Baseline) | −17.60(18.81) | −20.27(18.79) | −21.98(18.49) | −25.97(17.95) |
| p-Value | 1.83(3.52) | 0.332 | 0.140 | 0.002 |

p-Value = Difference relative to placebo, from Analysis of Covariance method (the center and baseline effect was removed)

Question #4: “Did you achieve enough lubrication to allow comfortable intercourse?” Sufficient lubrication for comfortable intercourse was improved by approximately 39% to 53% of the patients in the four groups vs. baseline. The difference between the groups was not statistically significant.

Question #5: “Did you achieve orgasm?” Being able to achieve orgasm was improved by approximately 22% to 42% vs. baseline. The difference between the groups was statistically significant.

Question #6: “What was your level of arousal?” The overall between-group difference was not statistically significant.

A total of 372 FSAD patients completed the study. Mean baseline parameters suggested no significant differences among treatment groups at the beginning. The primary efficacy as an improved mean arousal success rate from baseline, at the endpoint of the first treatment period, was 17.0%, 32.0%, 27.5% and 38.7% in the placebo, 0.5 mg PGE₁, 0.7 mg PGE₁, and 0.9 mg PGE₁ groups, respectively (p=0.0113, p=0.0855 and p=0.0006 vs. placebo). At the end of the second treatment period, the primary efficacy was further improved up to 28.6%, 41.9%, 40.3% and 51.4%, (p=0.0533, p=0.0735 and p=0.0002 vs. placebo). At the end of the entire treatment period, the primary efficacy was further improved up to 22.6%, 36.7%, 34.0% and 44.3%, (p=0.0224, p=0.0.0358 and p=0.0002 vs. placebo). The changes of the FSFI score relative to baseline, at the end of the first treatment period, were 10.0, 14.5, 15.7 and 18.2 in the placebo, 0.5 mg PGE₁, 0.7 mg PGE₁, and 0.9 mg PGE₁ groups. At the end of the second treatment period, the changes of the FSFI scores from baseline were further improved up to 14.7, 20.7, 21.7 and 22.9, in the placebo, 0.5 mg PGE₁, 0.7 mg PGE₁, and 0.9 mg PGE₁ groups, respectively. The changes of the FSDS scores relative to baseline, were −10.3, −14.7, −13.8 and −18.4 at the end of the first treatment period, and −17.6, −20.3, −22.0 and −26.0, at the endpoint of the second period with the placebo, 0.5 mg PGE₁, 0.7 mg PGE₁, and 0.9 mg PGE₁ groups, respectively.

These results demonstrate that the topical PGE₁ cream (0.9 mg PGE₁ group) significantly improved the sexual arousal rate of FSAD women during the first treatment period. The therapeutic effects of the PGE₁ cream were further enhanced after the second continuous dosing period. The efficacy of 0.9 mg PGE₁ group was better than the 0.5 mg and 0.7 mg PGE₁ groups. The results of FSFI and FSDS analysis showed a similar trend in support that the second continuous dosing period could further enhance the therapeutic effects of the PGE₁ creams.

In summary, the arousal success rate of each visit was calculated and statistically described. The rank-sum test was used to determine the difference between groups. The Primary Efficacy Analysis of the Intent-To-Treat (ITT) population showed a significant increase in arousal success rates with increasing dose. Arousal success rates at the end of the entire evaluation period were 33%, 46%, 43%, and 54% for placebo, 0.5 mg PGE₁, 0.7 mg PGE₁ and 0.9 mg PGE₁ treatment groups respectively. The Secondary Efficacy Analysis of the ITT population at the end of the second evaluation period showed a significant increase in FSFI scores versus placebo at the 0.9 mg PGE₁ doses. A significant decrease in distress while on the drug is reflected in the data, which shows significant differences from placebo at the 0.9 mg PGE₁ doses. This is an important finding since stress is an integral component of female sexual arousal disorder. The analysis of the Global Assessment Question scores—a measurement of the patient perceived improvement in arousal—shows a significant improvement versus placebo at all dose levels for PGE₁.

TABLE 28
Summary of Efficacy Results (ITT Population)

| | | 0.5 mg | 0.7 mg | 0.9 mg |
|---|---|---|---|---|
| | Placebo | PGE₁ | PGE₁ | PGE₁ |
| Number of patients | 97 | 91 | 91 | 95 |
| Success rate (%) | 33 | 46 | 43 | 54 |
| [FSEP Question 3] | | (p = 0.0162)* | (p = 0.040)* | (p < 0.0002)* |
| Mean change from | 14.7 | 20.7 | 21.7 | 22.9 |
| baseline in FSFI | | (p = 0.067)* | (p = 0.035)* | (p = 0.002)* |
| total score | | | | |
| Mean change from | −17.6 | −20.3 | −22 | −26.7 |
| baseline in FSDS | | (p = 0.332)* | (p = 0.140)* | (p = 0.002)* |
| total score | | | | |
| Global Assessment | 49 | 64 | 64 | 66 |
| Score (% reporting | | (p = 0.0339)* | (p = 0.0395)* | (p = 0.0166)* |
| improvement in arousal) | | | | |

value compared to placebo

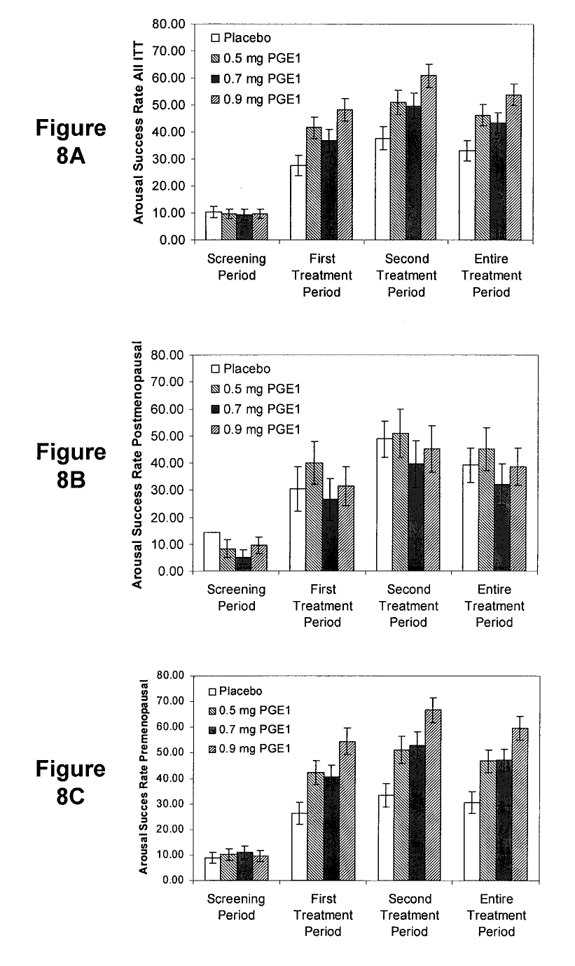

FIGS. 8A-8C summarize the results of the primary efficacy measure, arousal success rate for the four treatment groups at different stages of the study. FIG. 8A is a graphical representation of the mean arousal success data of Table 22, where the error bars are ±1 standard error of the mean for the four treatment groups at the screening period, for the first treatment period, for the second treatment period and for the entire treatment period for the total ITT population. FIG. 8B is a graphical representation of the mean arousal success data of Table 23, where the error bars are ±1 standard error of the mean for the four treatment groups at the screening period, for the first treatment period, for the second treatment period and for the entire treatment period for the postmenopausal patients. FIG. 8C is a graphical representation of the mean arousal success data of Table 24, where the error bars are ±1 standard error of the mean for the four treatment groups at the screening period, for the first treatment period, for the second treatment period and for the entire treatment period for the pre-menopausal patients. In addition to an increase in the arousal success rate associated with the repeated application of the placebo composition, there is a greater increase in the arousal success rate in the three alprostadil treatment groups between the two treatment periods as well as between the treatment periods and the screening period.

What is claimed:

1. A method of treatment for female sexual arousal disorder comprising the steps of:
providing a single dose of a topical prostaglandin composition comprising about 0.5 mg to about 1.5 mg prostaglandin E₁;
administering the single dose of the topical prostaglandin composition to the clitoris and the inner anterior wall of the vagina about 5 to about 20 minutes before intercourse; and
repeating the step of administering at least twice in six weeks.

2. The method of claim 1 wherein the single dose of the topical prostaglandin composition comprises about 0.5 mg to about 0.9 mg prostaglandin E₁.

3. The method of claim 1 wherein the step of administering is repeated at least three to five times in a month.
